(19) United States
(12) Patent Application Publication
(10) Pub. No.: US 2013/0028895 A1
(43) Pub. Date: Jan. 31, 2013

(54) EXOSOME INHIBITING AGENTS AND USES THEREOF

(71) Applicant: Gerald Wulf, Gottingen (DE)

(72) Inventor: Gerald Wulf, Gottingen (DE)

(21) Appl. No.: 13/192,220

(22) Filed: Jul. 27, 2011

(51) Int. Cl. A61K 39/395; A61P 17/06; A61K 38/00; A61K 31/7088; A61P 35/00; A61P 35/02

(52) U.S. Cl. 424/135.1; 424/130.1; 424/133.1; 514/1.1; 514/44 R

(57) ABSTRACT

The invention relates to methods and compositions for reducing exosome mediated tumor resistance against a therapeutic binding molecule and for increasing the efficacy of a therapeutic binding molecule suitable in the treatment of a disease. The methods include administering an effective amount of at least one agent inhibiting exosome formation and administering the therapeutic binding molecule.

### BACKGROUND OF THE INVENTION

Monoclonal antibody-based therapy has evolved as a mainstay of targeted anti-cancer therapy, endowing access of both direct and immunomediated lytic principles to the tumor cells. Anti-CD20 chimeric antibody rituximab was one of the first antibodies with high clinical efficacy, defining new standards of immunotherapy in malignant B-cell lymphoma.

Lymphomas are a group of malignant diseases originating from the lymphatic system. Lymphoma disease is derived from the malignant transformation of lymphatic cells of different maturation and differentiation stages. Depending on the type of cell, different lymphoma types may develop, which differ in susceptibility to therapy and prognosis. Traditionally, it is differentiated between the Hodgkin lymphoma (HL) and the heterogeneous group of the Non Hodgkin lymphomas (NHL). One type of aggressive NHL is the diffuse large B-cell lymphoma (DLBL), which accounts for approximately 40% of aggressive lymphomas among adults.

Standard treatment of DLBCL is a polychemotherapy such as to as the CHOP regimen. The addition of the monoclonal antibody rituximab (R-CHOP) improved survival and rates of complete responses for DLBL patients. R-CHOP is a combination of the monoclonal antibody (rituximab/Rituxan), 3 chemotherapy drugs (cyclophosphamide/Cytoxan, doxorubicin/Hydroxydaunorubicin, vincristine/Oncovine) and one steroid (prednisone). Radiation is another treatment modality used to consolidate localized disease control.

Current immunochemotherapy regimens can provide cure to significant proportions of patients with aggressive lymphoma, and prolong survival in patients with indolent B-cell lymphomas. However, the prognosis for patients with primary resistant or relapsed aggressive lymphoma is still dismal (recently reviewed in Gisselbrecht, C. Br. J. Haematol. 143, 607-621 (2008)).

Rituximab exerts its cytolytic effects after CD20 ligation by direct induction of apoptosis, complement-dependent cytolysis (CDC), as well as antibody-dependent cellular cytotoxicity (ADCC), with variation in the contribution to cytotoxicity, depending on the B-cell lymphoma entity. Independent of the mechanism, however, initiation of cytolysis always requires binding of the antibody to the tumor cell surface.

Exosomes are defined as microvesicular structures with a mean size of 50-100 nm, released by exocytosis following intracellular assembly in multivesicular bodies (MVB, review in Thery, C., Ostrowski, M., & Segura, E. Nat. Rev. Immunol. 9, 581-593 (2009)). In normal physiology, exosomes are secreted from erythroid progenitors during progenitor cell maturation, as well as from B-lymphocytes and dendritic cells, with multiple immune functions. Such immune functions, in particular the role of exosome as antigen presenting vesicles, have led to investigations into clinical applications, mostly aiming at a vaccination against malignant disease (Zitvogel, L. et al. Nat. Med. 4, 594-600 (1998); Escudier, B. et al. J. Transl. Med. 3, 10 (2005)). Exosomes have also been detected in the supernatant of several tumor cell lines, such as the T-lymphoblastic cell line Jurkat and the erythroleukemic cell line K562 (Bard, M. P. et al. Am. J. Respir. Cell Mol. Biol. 31, 114-121 (2004); Savina, A., Fader, C. M., Damiani, M. T., & Colombo, M. I. Traffic. 6, 131-143 (2005)).

It has recently been discovered that the intracelluar compartment of exosome assembly, i.e. the multivesicular bodies (MVBs), is modulated by the ABC transporter A3 in hematological neoplasm with myeloid differention, associated with resistance against a broad spectrum of cytostatic drugs (Chapuy, B. et al. Leukemia 22, 1576-1586 (2008); Steinbach, D. et al. Clin. Cancer Res. 12, 4357-4363 (2006); Chapuy, B. et al. Haematologica 94, 1528-1536 (2009)). In addition to its role in leukemia, ABCA3 levels were also detected in aggressive lymphoma even exceeding those in myeloid leukemia (Chapuy, B. et al. (2008), supra). To date, ABC transporters have not been recognized to modulate exosome biogenesis. Beyond surfactant biogenesis, it has recently been discovered that leukemia cells express ABCA3, and that their expression is associated with decreased susceptibility to cytostatic therapy, mediated through cytoplasmatic sequestration of the cytocidal substances (Efferth, T. et al. Mol. Cancer Ther. 5, 1986-1994 (2006); Chapuy, B. et al. Leukemia 22, 1576-1586 (2008); Steinbach, D. et al. Clin. Cancer Res. 12, 4357-4363 (2006); Chapuy, B. et al. Haematologica 94, 1528-1536 (2009)). Interestingly, ABCA3 appears to protect tumor cells against some of the most efficient cytostatic drugs applied in lymphoma therapy, i.e. vincristine, anthracyclines, and etoposide (Chapuy, B. et al. (2008), supra).

Indometacin had been shown to suppress the expression of ABCA3 in acute myeloid leukemia cells, restoring susceptibility to cytostatic therapy by downregulation of ABCA3 transcription (Song, J. H., Kim, S. H., Kim, H. J., Hwang, S. Y., & Kim, T. S. Int. J. Oncol. 32, 931-936 (2008)).

Exosomes, particularly those derived from dendritic or cancer cells, are under consideration as immunomodulators for cancer immunotherapy, e.g. exploiting exosomes as carriers of tumor-associated MHC-peptide complexes to antigen presenting cells. Further, WO 2010/056337 describes the use of exosomes in the diagnosis of cancer.

For some substances with an effect on exosome release, in particular rapamycin, a synergism in cytocidal efficacy together with anti-CD20 antibody-mediated lysis had already been described, albeit explained by alternative mechanisms associated with mTOR inhibition (Wanner, K. et al. Br. J. Haematol. 134, 475-484 (2006)).

Tumor cell susceptibility to immunochemotherapy still varies, with mostly fatal outcome in cases of resistant disease. Accordingly, there still remains the need for novel anti-cancer treatments and cancer therapeutics, in particular for the treatment of certain resistant cancers.

### SUMMARY OF THE INVENTION

The inventors analyzed exosome release from B cell lymphomas, and found strong exosome production and release from aggressive B-cell lymphoma cells in vitro and in vivo. B-cell lymphoma cells released exosomes which carried CD20, bound therapeutic anti-CD20 antibodies, consumed complement, and protected target cells from antibody attack. Such exosomes acted as decoy targets upon rituximab exposure, allowing lymphoma cells to escape from humoral immunotherapy. ABCA3, previously shown to mediate resistance to chemotherapy, was critical for the amounts of exosomes released and both pharmacological blockade and the silencing of ABCA3 enhanced susceptibility of target cells to antibody-mediated lysis.

The evidence in this disclosure has a focus on antibody binding and complement consumption. As the other effector mechanisms of rituximab, i.e. induction of apoptosis and ADCC, also depend on plasma membrane binding of rituximab, the inventors extrapolate that such cytocidal mechanisms may as well be perturbed by CD20-positive exosomes, both by exosomes as decoy targets and by the reduction of antibody targets on the tumor cells.

Thus, the present inventors are the first to show that lymphoma exosomes shield target cells from antibody attack and that exosome biogenesis is modulated by the lysosome-related organelle associated ABC transporter A3 (ABCA3).

Accordingly, in a first aspect, the invention relates to a method for reducing exosome mediated tumor resistance against a therapeutic binding molecule, the method comprising the steps of
(i) administering an effective amount of at least one agent inhibiting exosome formation, and
(ii) administering said therapeutic binding molecule,
wherein step (i) is conducted before or concomitant to step (ii).

In the method, the therapeutic binding molecule may be an antibody molecule, a polypeptide, peptide, peptidomimetic, or a small molecule having a molecular weight in the range of 250-800 Da. In the method, the at least one agent inhibiting exosome formation may be capable of perturbing multivesicular body (MVB) biogenesis, and/or perturbing membrane cholesterol supply. The at least one agent inhibiting exosome formation may be an inhibitor of a protein of the group A of ABC transporters. The at least one agent inhibiting exosome formation may be an inhibitor of phosphatidylinositol-3-kinase, or an inhibitor of ADAM-metalloproteases, or a calcium chelator. In the method, the therapeutic binding molecule may be directed against CD20, CD40, CD19, CD23, EpCAM, or CD37.

In another embodiment, the at least one agent inhibiting exosome formation and a therapeutic antibody may be formulated in a pharmaceutical composition.

In a second aspect, the invention relates to a method of increasing the efficacy of a therapeutic binding molecule suitable in the treatment of a disease, the method comprising the steps of
(i) administering an effective amount of at least one agent inhibiting exosome formation, and
(ii) administering said therapeutic binding molecule,
wherein step (i) is conducted before and/or concomitant to step (ii),
with the provisio that if the binding molecule is Rituximab, the exosome formation inhibiting agent is not rapamycin.

The disease may cancer, such as lymphoma, haematological cancers, chronic lymphocytic leukaemia (CLL), CTCL, lung cancer, ovarian cancer, prostate cancer, and breast cancer. The diseases may be a proliferative autoimmune disease. In the method, the therapeutic binding molecule may be an antibody molecule, a polypeptide, peptide, peptidomimetic, or a small molecule having a molecular weight in the range of 250-800 Da. The at least one agent inhibiting exosome formation may be capable of perturbing multivesicular body (MVB) biogenesis and/or perturbing membrane cholesterol supply. In the method, the therapeutic binding molecule may be an antibody molecule, selected from the group consisting of a polyclonal antibody, a monoclonal antibody, a recombinant full antibody (immunoglobulin), a F(ab)-fragment, a F(ab)2-fragment, a F(v)-fragment, a single-chain antibody, a chimeric antibody, a CDR-grafted antibody, a bivalent antibody-construct, a synthetic antibody, a cross-cloned antibody, a fully-human antibody, a humanized antibody, nanobodies, diabodies, or peptide aptamers.

The preferred embodiments of the first and second aspect of the invention are described in the following.

### BRIEF DESCRIPTION OF THE DRAWINGS

The invention may be understood with reference to the following drawings and description.

FIGS. 1A-D depict binding of therapeutic anti-CD20 monoclonal antibody to exosomes from B-cell lymphoma cells (A); Western blot confirming findings in A (B); detection of the CD20 target protein and exosome markers alix and flotillin-2 (flot.-2, C); and exosomal binding of therapeutic monoclonal antibodies rituximab and GA101, as well as of CD20, flotillin-2, CD63 and CD9 were also documented by FACS with detection of binding to purified exosome-labelled beads (D, here Su-DHL-4).

FIGS. 2A-F depict absorption of anti-CD20 antibody rituximab and consumption of complement on lymphoma-derived exosomes in vitro and in vivo

FIGS. 3A-D depict rescue of lymphoma cells from rituximab-mediated CDC by exosomes.

FIGS. 4A-C depict rituximab stimulated shedding of exosomal bound TCC from lymphoma cells.

FIGS. 5A-C depict inhibition of exosome shedding and enhanced CDC susceptibility induced by rapamycin, indometacin and U18666A.

FIGS. 6A-F depict role of ABCA3 for exosome release and anti-CD20 mediated complement dependent cytolysis.

FIGS. 7A-D depict morphology, purity and characterization of exosome preparations from aggressive B-cell lymphoma cell lines and patient samples.

FIGS. 8A-B depict binding of therapeutic antibody rituximab to exosomes in vivo.

FIG. 9 depicts a graph illustrating no absorption of anti-CD20 antibody rituximab by CD20 negative exosomes derived from K562 cells.

FIGS. 10A-B depict flow cytomerty grafts for cell line Balm-3 (A) and Su-DHL-4 (B) illustrating resistance of lymphoma exosomes against rituximab-initiated CDC.

FIGS. 11A-B depict graphs illustrating inhibition of exosome shedding from lymphoma cells.

FIG. 12 depicts a schematic illustration of the mechanisms involved in exosome-mediated protection of lymphoma cells from CDC attack.

### DETAILED DESCRIPTION OF THE INVENTION

This disclosure provides in vitro and in vivo evidence for the release of exosomes from e.g. B-cell lymphoma cells, the expression of a target of a therapeutic antibody thereon, such as CD20, the protection of lymphoma cells from the effects mediated by said therapeutic binding molecules, such as antibodies, e.g. rituximab-mediated CDC, and the regulatory role of exosome inhibiting agents. In this context, agents inhibiting intracellular ABC transporter A3 and thus exosome release may provide for new combination treatments along with therapeutic antibodies directed against epitopes associated with a specific disease, such as cancer, if said epitope can be found on such exosomes.

The results shown herein add to the understanding of target cell resistance against humoral immunotherapy. This general concept of exosome-mediated resistance against therapeutic binding molecules, such as antibodies, directed against surface molecules, which can be detected on such exosomes, is exemplified herein with the therapeutic antibody rituximab, which is directed against CD20. The data shows that CD20 participates in plasma circulation as a protein embedded in the exosomal membrane that intercepts therapeutic antibodies directed against CD20, such as rituximab. Such epitope-specific binding of anti-CD20 antibody outside the plasma membrane was previously not appreciated, since neither the transmembrane CD20 protein, nor antigenic parts thereof circulate as free molecules.

Although the examples are mainly directed to therapeutic antibodies, it is evident to the skilled person that the present teaching can be generalized to any therapeutic binding molecule, which is directed to a target, which can be found on exosomes, and is thus susceptible to exosome mediated resistance.

Accordingly, the invention relates to at least one agent inhibiting exosome formation, for use in the treatment of cancer in a patient which acquired or may acquire exosome mediated resistance against a therapeutic binding molecule suitable for treating said cancer, wherein the at least one exosome formation inhibiting agent is administered before and/or concomitant with said therapeutic binding molecule.

The term “at least one” agent means one or more than one agent, such as two, three, four, five, six, seven, eight, nine or ten different agents.

Exosome formation and inhibition of exosome formation can be measured, for example, by differential centrifugation according to standard protocols (modified according to Valadi, H. et al. Nat. Cell Biol. 9, 654-659 (2007)). Following incubation of 5×10⁷ cells of interest for 48 h in complete exosome-free medium, cells and larger debris are removed by centrifugation for 10 min (10 min., 500 g, 4° C.). The supernatant is centrifuged again (20 min., 10000 g; 4° C.; Beckman L8-55 ultracentrifuge, rotor Ti32) to remove intermediate size particles. Subsequently the supernatant containing exosomes is filtered (0.22 μM Millex GP), and again centrifuged (240 min., 120 000 g, 4° C.; Beckman L8-55 ultracentrifuge, rotor Ti32) to obtain the exosome pellet, which is washed once in PBS, and finally re-suspended in 50 μl PBS for further applications. Exosomes are quantified by measuring whole protein according to standard protocols (BioRad-DC-Protein-Assay), Western blot detecting flotillin-2 in comparison with whole cells or control exosome preparations, and acetyl-cholin-esterase (AChE) activity as previously described (Savina, A., Fader, C. M., Damiani, M. T., & Colombo, M. I. Traffic. 6, 131-143 (2005); incorporated herewith by reference).

Flotillin is a cytosolic, membrane-associated protein involved in scaffolding functions, signaling and endocytosis. Its two isoforms, flotillin-1 and flotillin-2, are enriched in exosomes and can be used as protein markers to quantify exosomal release (Trajkovic et al. J. Cell Biol. 172, 937-948 (2006), Strauss, K. et al. J. Biol. Chem. 285(34), 26279-26288 (2010), both incorporated herewith by reference).

Inhibition of exosome formation can be measured by comparison of cells incubated with the at least one exosome formation inhibiting agent and corresponding cells which have been cultured without the at least one exosome formation inhibiting agent. Preferably, said assay is performed by using several cultivations using different concentrations of the exosome formation inhibiting agent. The skilled person will know how to determine the optimal concentration of the exosome formation inhibiting agent.

Preferably, the exosome formation in the cells incubated with the exosome formation inhibiting agent is at most 90% as compared to a corresponding cell not incubated with the exosome formation inhibiting agent, when cultured under otherwise comparable conditions. More preferably, the exosome formation in the cells incubated with the exosome formation inhibiting agent is at most 80%, such as at most 75%, even more preferably at most 70%, e.g. at most 65%, or at most 60%, still even more preferably at most 55%, such as at most 50%, or at most 45%, and most preferably at most 40%, such as at most 35% or even at most 30%, at most 25%, or 20% as compared to a corresponding cell not incubated with the exosome formation inhibiting agent, when cultured under otherwise comparable conditions.

In this context, the term “exosome mediated resistance” is to be understood as a mechanism of a cell to escape the action and/or effect of a therapeutic binding molecule by formation of exosomes, which carry the target of the binding molecule, and which will therefore consume a considerable amount of the therapeutic binding molecule. As a consequence, these exosome-bound binding molecules are no longer capable of carrying out their intended function.

Further, the term “acquired” exosome mediated resistance is intended to mean that the cancer or other disease was not originally resistant against the therapeutic binding molecule, e.g. the therapeutic antibody. For example, in contrast thereto, Wanner et al. discloses the use of Rituximab and rapamycin, but for the treatment of cells, which have a defect in the signalling of CD20, and thus are originally resistant against therapeutic binding molecules directed against CD20.

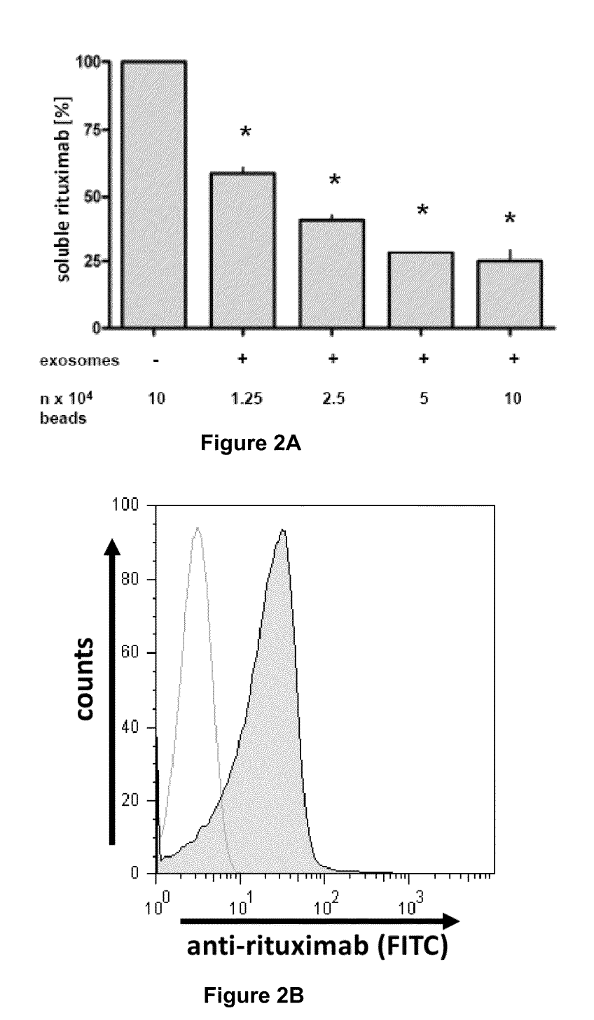

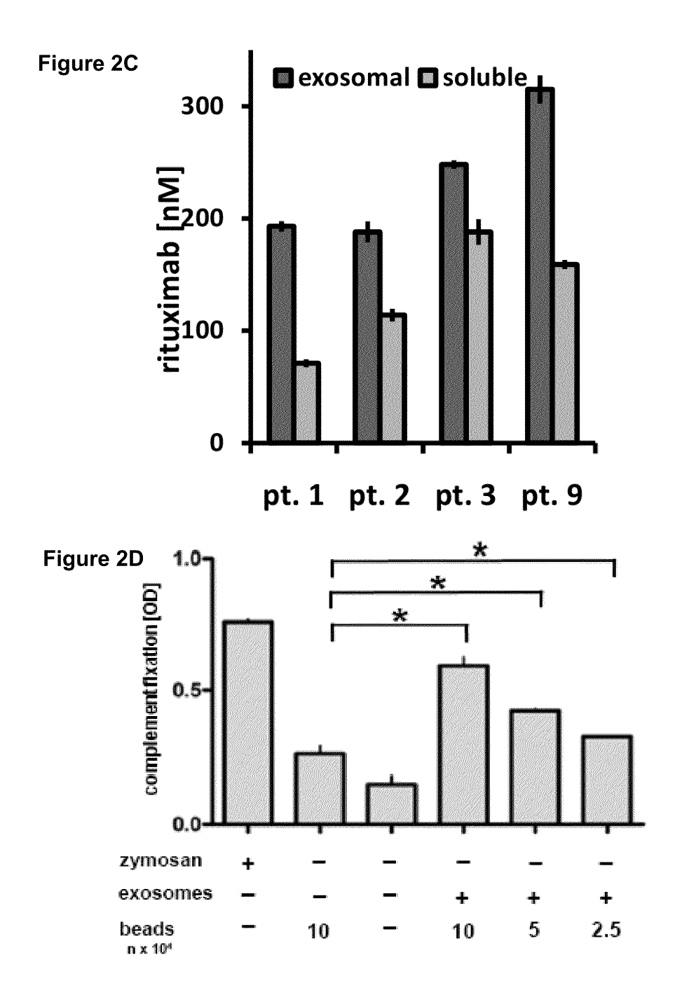

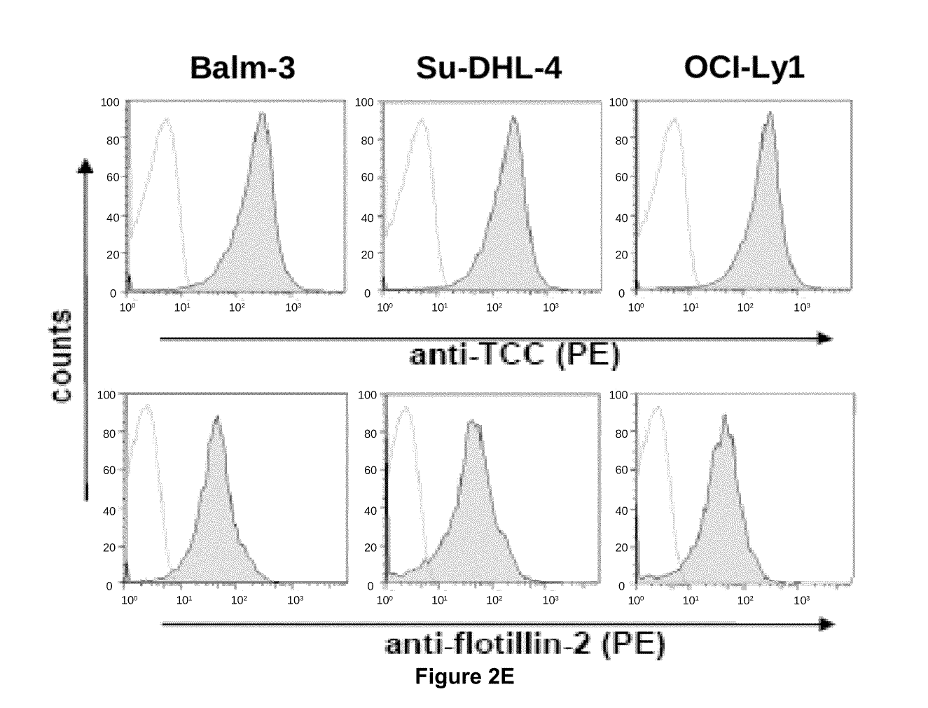

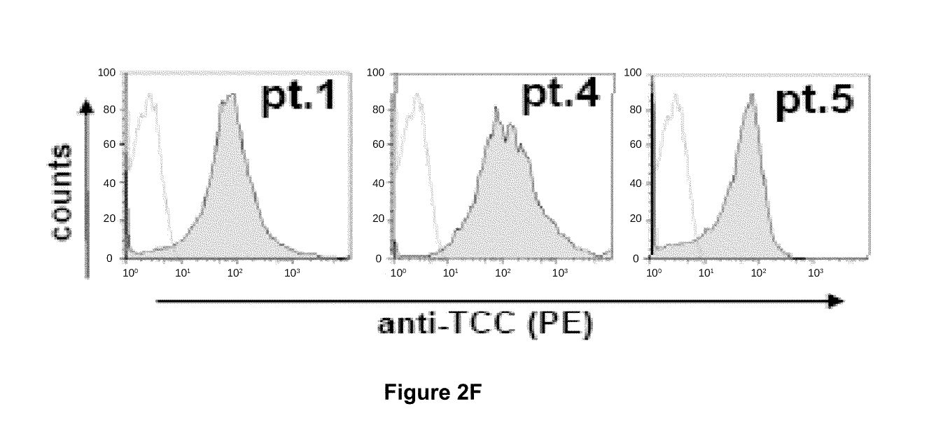

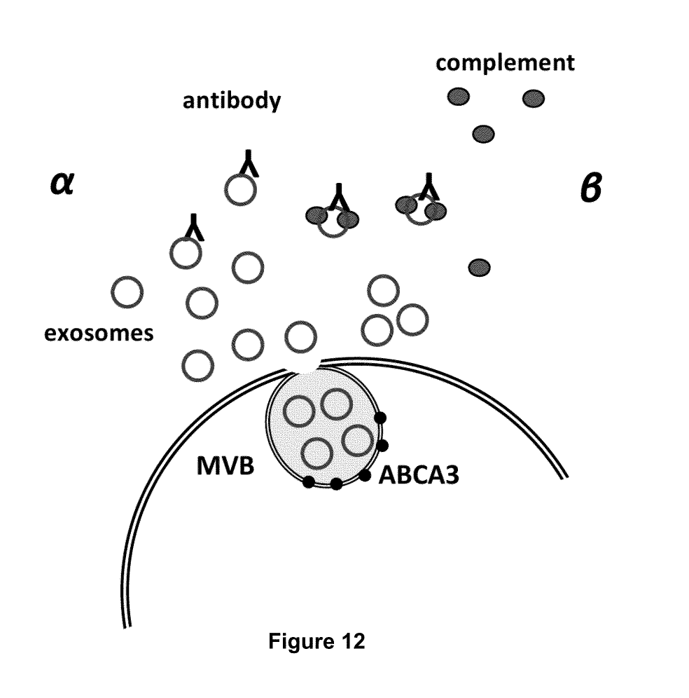

With regard to rituximab, as shown in the examples, this means that rituximab bound to exosomes is not available for plasma membrane attack, thus limiting the availability of cytocidal antibody in situations of high tumor—and tumor exosome—load, i.e. at the start of therapy. Noteworthy, the currently available assay system for rituximab pharmacokinetics using the anti-idiotypic antibody MB2A4 detects both soluble and—at high proportions—exosomal bound antibody (see FIG. 2). The inventors' findings correlate with the clinical observation that starting rituximab antibody therapy requires high doses of antibody to achieve efficient plasma levels. In this situation the tumor mass itself certainly binds high amounts of rituximab, but the absorption of rituximab into circulating exosomes may further contribute to this initial “sink” effect. Thus exosomal CD20 represents a decoy target for rituximab, reducing the number of antibody molecules effectively reaching the tumor cell (overview in FIG. 12).

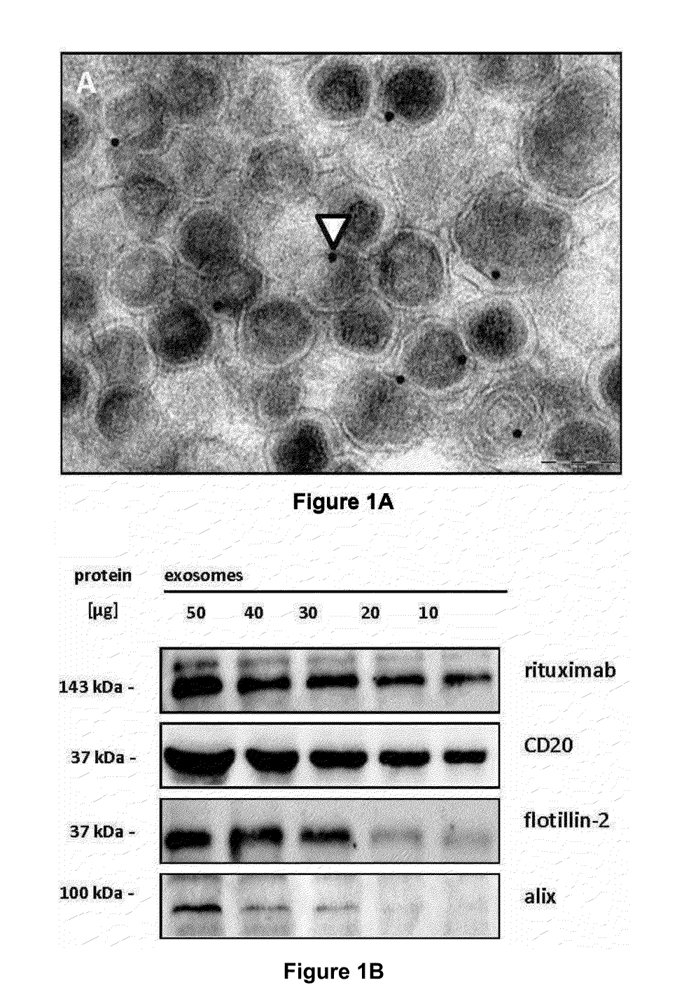

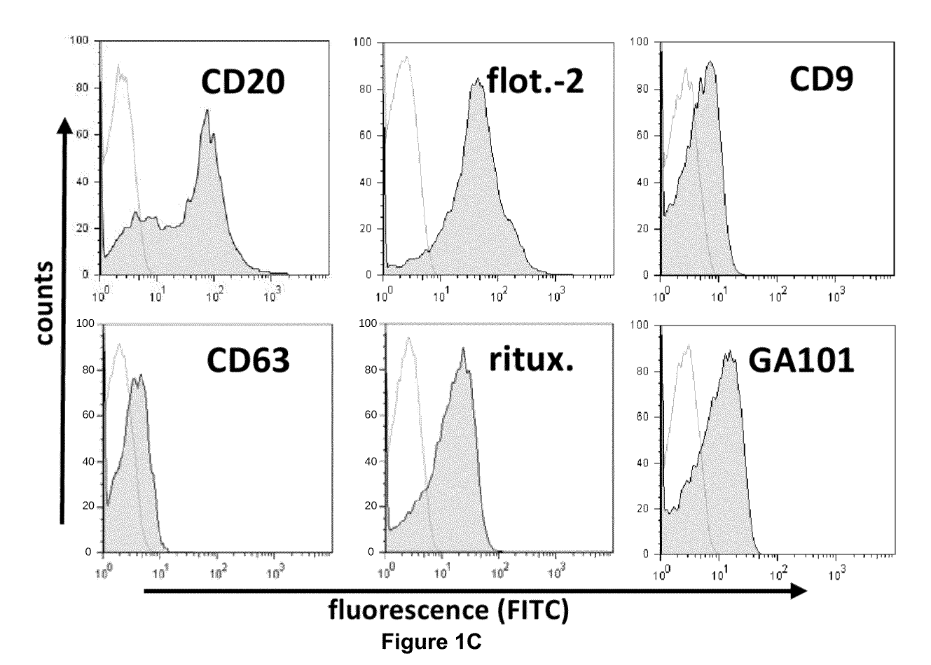

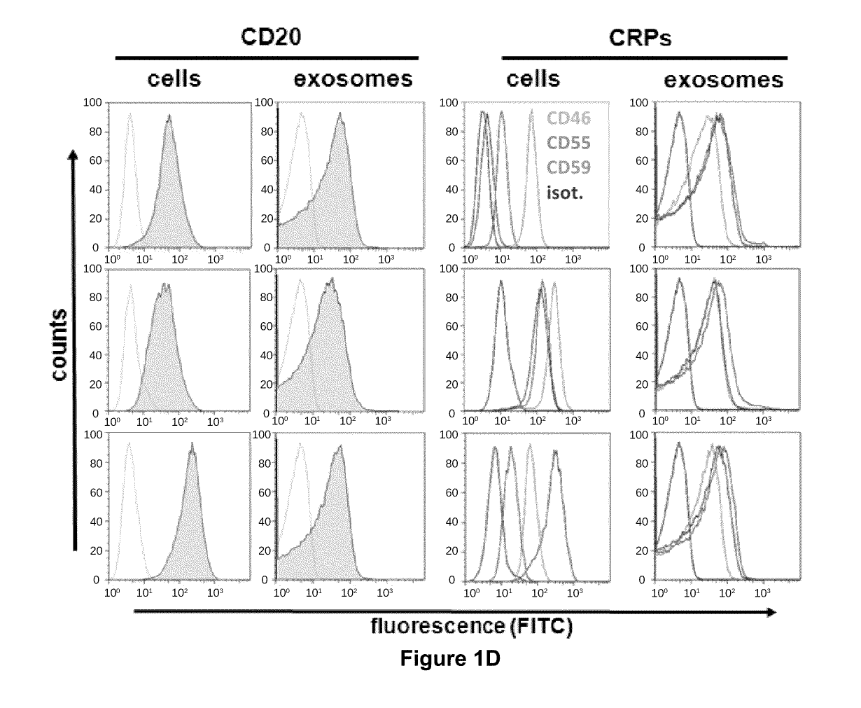

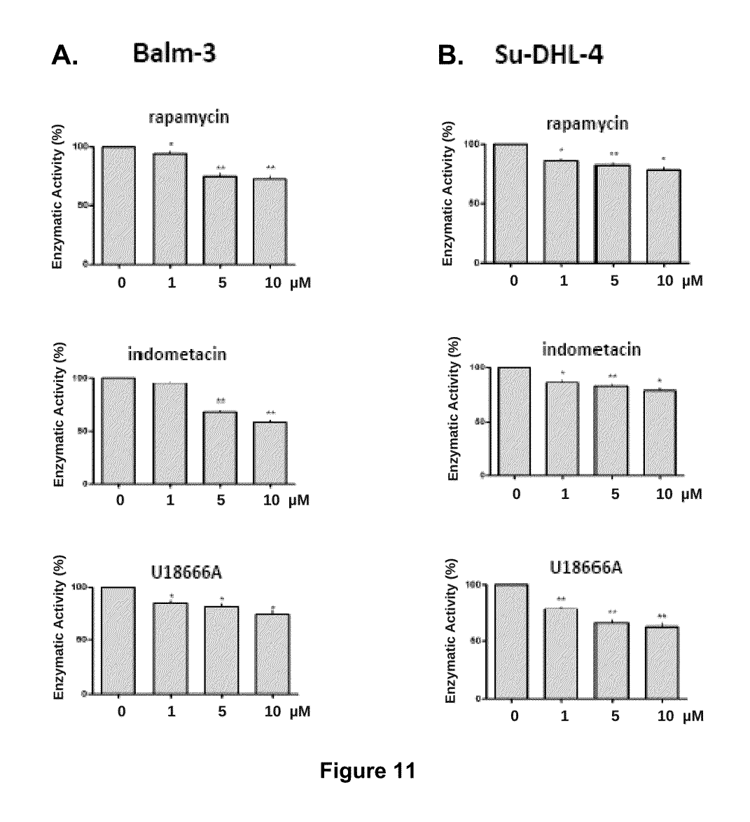

In addition to antibody consumption, the data herein clearly demonstrate that rituximab bound to lymphoma exosomes fixes complement in vitro and in vivo, and that the consumption of complement impedes the efficacy of rituximab-initiated CDC. Complement consumption occurs in the circulation, but local depletion of complement in the tumor micromilieu may even be of more relevance in vivo. Studies of tumor tissue sections have previously documented complement deposition to occur predominantly in proximity to intratumoral blood vessels, where the ratio of antibody and complement passes an optimum for fixation and cell lysis. Given the accumulation of exosomes in the interstitial space (FIG. 7), exosomal complement consumption may contribute to the protection of tumor cells particularly at a distance from the blood vessels. It is noted that the inventors found that lymphoma-derived exosomes dispose of high amounts of complement regulating proteins (CRPs), protecting the microvesicles themselves from complement mediated lysis (FIG. 1, FIG. 11), a finding also reported for exosomes from normal B-cells and antigen presenting cells. Furthermore, the inventors found sublytic antibody attack to enhance secretion of exosomes. Thus, the protective effects of lymphoma-derived exosomes represent both a constitutive property of the tumor cells, and a resistance mechanism recruitable as an adaptative response to CDC-associated cellular stress.

However, CD20 and its cognate therapeutic antibody rituximab is merely to be understood as one example. The data shown herein makes it plausible that also other targets of therapeutic binding molecules, such as therapeutic antibodies, may escape e.g. cytolysis by an exosome mediated resistance mechanism.

In another aspect, the invention relates to at least one agent inhibiting exosome formation, for use in the treatment of a disease in a patient which acquired or may acquire exosome mediated resistance against a therapeutic binding molecule suitable for treating said disease, wherein the at least one exosome formation inhibiting agent is administered before and/or concomitant with said therapeutic binding molecule, with the provisio that if the antibody is Rituximab, the exosome formation inhibiting agent is not rapamycin, in particular if the disease is a cancer.

Generally, the present invention includes treatment of diseases, which involve increased growth or ‘unintended’ survival of cells. Although the present invention is not to be limited to cancer only, one example of such a disease is cancer. Thus, in a preferred embodiment, the disease is cancer, preferably a cancer which acquired or may acquire exosome mediated resistance against a therapeutic binding molecule suitable for treating said cancer. In particular, and as demonstrated in the examples, the present invention is useful in the treatment of lymphoma, e.g. Non-Hodgkins lymphoma, Hodgkin's lymphoma, and follicular lymphoma, and haematological cancers. However, it is contemplated that the present invention is also useful in the treatment of other cancers.

For example, it was shown in Buschow et al. Immunology and Cell Biology (2010) 88, 851-856 (which is herewith incorporated by reference), that in addition to CD20, cell surface markers such as CD40, CD19, CD23 (Fc epsilon R2), CD37 are present on exosomes. Likewise, EpCam may also be present on exosomes. These surface markers are typically found or therapeutic targets in chronic lymphocytic leukaemia (CLL); CTCL; lung cancer, e.g. non-small cell lung carcinoma; ovarian cancer; prostate cancer; and breast cancer. Hence, in a preferred embodiment, the cancer to be treated is selected from the group of cancers consisting of lymphoma, e.g. Non-Hodgkins lymphoma, Hodgkin's lymphoma, and follicular lymphoma; haematological cancers; chronic lymphocytic leukaemia (CLL); CTCL; lung cancer, e.g. non-small cell lung carcinoma; ovarian cancer; prostate cancer; and breast cancer. Preferably, the cancer is lymphoma, such as Non-Hodgkins lymphoma, Hodgkin's lymphoma, or follicular lymphoma; or haematological cancer; chronic lymphocytic leukaemia (CLL); CTCL; or lung cancer, e.g. non-small cell lung carcinoma. More preferably the cancer is lymphoma, such as Non-Hodgkins lymphoma, Hodgkin's lymphoma, or follicular lymphoma; or haematological cancer, even more preferably the cancer is lymphoma, such as Non-Hodgkins lymphoma, Hodgkin's lymphoma, or follicular lymphoma. Most preferably the cancer is lymphoma, e.g. Non-Hodgkins lymphoma. However, it is also contemplated that the present invention is useful in the treatment of further cancers, which involves the use of therapeutic binding molecules, such as therapeutic antibodies. Cancers which involve such a treatment are neuroblastoma; colorectal cancer; gastrointestinal cancer; squamous cell carcinoma; head and neck cancer; nasopharyngeal cancer; pancreatic cancer; and melanoma.

Most preferably, the therapeutic binding molecule is the therapeutic antibody Rituximab and the disease is lymphoma, leukemia, or haematological cancer, more preferably a Non-Hodgkin-Lymphom.

However, it is contemplated that the invention is useful in the treatment of any disease, in which an exosome mediated resistance can develop. Therefore, it is contemplated that the present invention is also useful in the treatment of a proliferative autoimmune disease, which acquired or may acquire exosome mediated resistance against a therapeutic binding molecule suitable for treating said disease, e.g. in the treatment of psoriasis. There are five types of psoriasis: plaque, guttate, inverse, pustular and erythrodermic. The most common form, plaque psoriasis, is commonly seen as red and white hues of scaly patches appearing on the top first layer of the epidermis, which is caused by a rapid accumulation of skin at these sites.

The present invention may also be useful in treating other autoimmune diseases, which acquired or may acquire exosome mediated resistance against a therapeutic binding molecule suitable for treating said autoimmune disease. For example, it is contemplated that the present invention may be useful in treating rheumatois arthritis, Crohn's disease, ulcerative colitis, acute rejection of (kidney) transplants, and/or allergic asthma.

A “patient” as used herein is, may be a non-human patient or a human. Preferably, the patient is a mammal such as a horse, cow, pig, mouse, rat, guinea pig, cat, dog, goat, sheep, non-human primate, or a human.

The term “binding molecule”, as used herein, is intended to refer to any kind of therapeutic molecule that binds with high affinity to its cognate target. For example, the binding molecule may be an antibody molecule, a polypeptide, peptide, peptidomimetic, or a small molecule having a molecular weight in the range of 250-800 Da, preferably in the range of 300 to 750 Da, such as 350 to 700 Da, or 400 to 650 Da.

Accordingly, the binding molecule may be a natural or synthetic peptide. The synthetic peptide or peptidomimetic may comprise natural or synthetic amino acids, such as standard and non-standard amino acids, including their respective D- and L-forms, unnatural amino acids as well as chemically modified amino acids.

A peptidomimetic is a small protein-like chain designed to mimic a peptide. Peptidomimetics may either be derived from modification of an existing peptide, or by designing similar systems that mimic peptides, such as peptoids and β-peptides. Thus, peptidomimetics may include organic compounds comprising a peptide backbone. Irrespective of the approach, the altered chemical structure of a peptidomimetic, such as altered backbones and the incorporation of non-natural amino acids, is designed to advantageously adjust the molecular properties, e.g. the stability or biological activity.

The small molecule may either be isolated from a natural source or developed synthetically, e.g., by combinatorial chemistry. Examples of such a small molecule include, but are not limited to synthetic compounds, as well as modifications of existing compounds. Also encompassed by the term small molecule are saccharide-, lipid-, peptide-, polypeptide- and nucleic acid-based compounds.

Peptide aptamers are proteins that are designed to interfere with other protein interactions inside cells. They consist of a variable peptide loop attached at both ends to a protein scaffold. This double structural constraint greatly increases the binding affinity of the peptide aptamer to levels comparable to an antibody's (nanomolar range).

In one particularly preferred embodiment, the binding molecule is an antibody molecule, selected from a polyclonal antibody, a monoclonal antibody, a recombinant full antibody (immunoglobulin), a F(ab)-fragment, a F(ab)₂-fragment, a F(v)-fragment, a single-chain antibody, a chimeric antibody, a CDR-grafted antibody, a bivalent antibody-construct, a synthetic antibody, a cross-cloned antibody, a fully-human antibody, a humanized antibody, nanobodies, diabodies, and peptide aptamers and therelike.

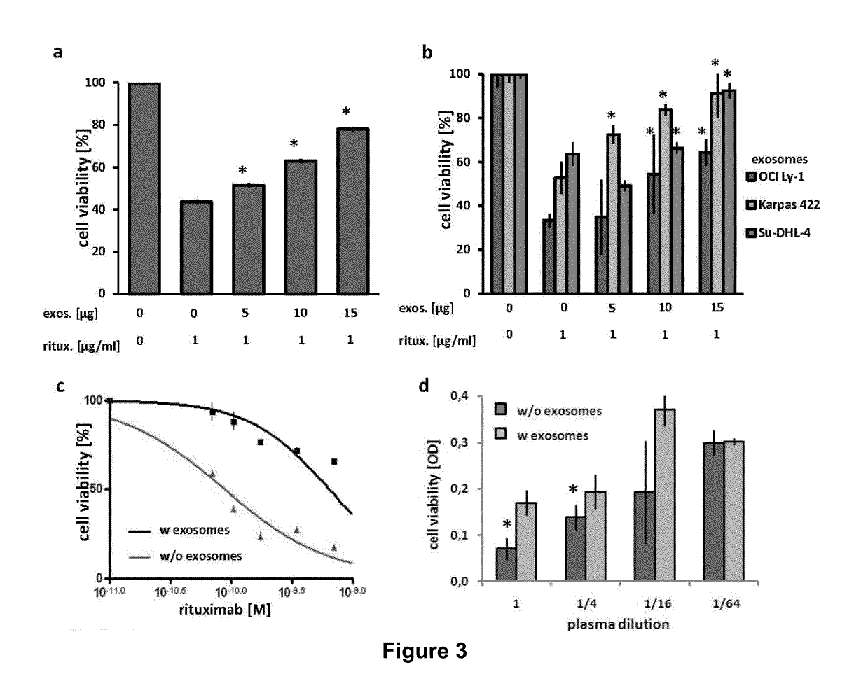

Antibodies or immunoglobulins are gamma globulin proteins consisting in their natural form of two large heavy chains and two small light chains linked by disulfide bonds (c.f. FIG. 3). There are five types of mammalian Ig heavy chain: α, δ, ε, γ, and μ. The type of heavy chain present defines the class (isotype) of the antibody; these are IgA, IgD, IgE, IgG, and IgM antibodies, respectively. Each heavy chain has two regions, the constant region and the variable region. The constant region is nearly identical in all naturally occurring antibodies of the same isotype of the same species. A light chain also consists of one constant domain and one variable domain. In mammals there are two types of immunoglobulin light chain, lambda (λ) and kappa (κ).

Although the general structure of all antibodies is very similar, the unique property of a given antibody is determined by the variable (V) regions. More specifically, variable loops, three each the light (V_{L}) and three on the heavy (V_{H}) chain, are responsible for binding to the antigen, i.e. for its antigen specificity.

With regard to the term “full antibody”, any antibody is meant that has a typical overall domain structure of a naturally occurring antibody (i.e. comprising a heavy chain of three or four constant domains and a light chain of one constant domain as well as the respective variable domains), even though each domain may comprise further modifications, such as mutations, deletions, or insertions, which do not change the overall domain structure.

In addition, the term “antibody” is intended to comprise all above-mentioned immunoglobulin isotypes, i.e. the antibody may be an IgA, IgD, IgE, IgG, or IgM antibody, including any subclass of these isotypes. Preferably, the antibody is an IgG antibody, more preferably an IgG1 or IgG2 antibody. Since the antibody may be expressed and produced recombinantly, the antibody may also comprise two different constant regions of heavy chains, e.g. one IgG1 and one IgG2 heavy chain, or heavy chains from different species. However, the heavy chains are preferably from the same species, more preferably from the species of the patient. Moreover, the antibody comprises either a lambda or a kappa light chain.

An “antibody fragment” also contains at least one antigen binding fragment as defined above, and exhibits the same function and specificity as the complete antibody of which the fragment is derived from. Fab fragments can be generated by using the enzyme papain to cleave an immunoglobulin. The enzyme pepsin cleaves below the hinge region and, thus, below the disulfide bonds, so that an F(ab)₂ fragment is formed. Moreover, the variable regions of the heavy and light chains can be fused together to form a single chain variable fragment (scFv). Thus, in the context of the this invention, an antibody also comprises variable and light regions, F(ab)-, F(ab)₂ fragments, CDR-regions, etc. Such “fragments” are known in the art and can readily be used in recombinant technologies. The antibodies referred to herein, also comprise humanized or CDR-grafted antibodies as well as genetically/recombinantly engineered “full human” antibodies. Such an engineered antibody is for example an antibody, in which at least one region of an immunoglobulin of one species is fused to another region of an immunoglobulin of another species by genetic engineering in order to reduce its immunogenicity. Also provided are derivatives of antibodies, like single-chain antibodies, diabodies, bispecific single chain antibodies, and antibody-like molecules, such as peptide aptamers and the like.

Usually, therapeutic antibodies are monoclonal antibodies. The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be constructed as requiring production of the antibody by any particular method.

In a particularly preferred embodiment, the therapeutic antibody is a cytolytic antibody, preferably wherein the antibody (i) directly induces apoptosis, and/or (ii) mediates complement-dependent cytolysis, and/or (iii) mediates antibody-dependent cellular cytotoxicity.

As noted above, it was shown in Buschow et al., that in addition to CD20, cell surface markers such as CD40, CD19, CD23 (Fc epsilon R2), CD37 are present on exosomes. EpCam may also be present on exosomes. Accordingly, in one preferred embodiment, the therapeutic binding molecule, e.g. the therapeutic antibody, is directed against CD20, CD40, CD19, CD23, CD37, or EpCAM, more preferably against CD20, CD40, CD19, CD23, or CD37, and most preferably the therapeutic binding molecule, e.g. the therapeutic antibody, is directed against CD20.

Suitable therapeutic antibodies as described above are known to the skilled person. Accordingly, in a preferred embodiment, the therapeutic antibody is selected from the group of therapeutic antibodies consisting of Rituximab, Afutuzumab, Ibritumomab tiuxetan, Ofatumumab, Tositumomab, Veltuzumab, Blinatumomab, Dacetuzumab, Lucatumumab, Lumiliximab, Taplitumomab paptox, Adecatumumab, Catumaxomab, Edrecolomab, Oportuzumab monatox, Tucotuzumab celmoleukin, Efalizumab, Inolimomab, preferably wherein the therapeutic antibody is selected from Rituximab, Ibritumomab tiuxetan, Ofatumumab, Tositumomab, and Veltuzumab, most preferably wherein the therapeutic antibody is Rituximab.

Nevertheless, exosomes may also carry further membrane-bound surface markers of therapeutic relevance. Such surface marker include VEGFR2, GD2, CA-125, CD52, CEA, TAG-72, BAFF, VEGF-A, CD44, CD30, mucin CanAg, IGF1R, MUC1, TRAIL-R2, SLAMf7, CD22, HER2/neu, integrin αvβ3, folate receptor 1, TGF-β, GPNMB, CD51, CD152, CD33, CD56, EGFR, CD74, C242 antigen, 5T4, PDGF-Rα, VEGFR2, HGF, FAP, tenascin C, CTLA-4, IL-13, or integrin α5β1, TNF-R1, TNF-R2, IL-2R alpha (CD25), IL-2R beta (CD122), Fc epsilon R1, and CD11a. Therefore, the therapeutic binding molecule, e.g. the therapeutic antibody, may be directed against any one of these surface markers.

Therapeutic antibodies which are directed against these surface markers, and which have been found or are tested for the treatment of cancer or a proliferative immune disease are known to the skilled person, and include, but are not limited to 3F8, Abagovomab, Alacizumab pegol, Alemtuzumab, Anatumomab, Apolizumab, Belimumab, Bevacizumab, Bivatuzumab, Brentuximab vedotin, Cantuzumab mertansine, Cetuximab, Citatuzumab bogatox, Cixutumumab, Clivatuzumab tetraxetan, Conatumumab, Detumomab, Ecromeximab, Elotuzumab, Epratuzumab, Ertumaxomab, Etaracizumab, Farletuzumab, Fresolimumab, Glembatumumab vedotin, Intetumumab, Inotuzumab ozogamicin, Ipilimumab, Iratumumab, Labetuzumab, Lexatumumab, Lintuzumab, Lorvotuzumab mertansine, Mapatumumab, Matuzumab, Milatuzumab, Mitumomab, Nacolomab tafenatox, Naptumomab estafenatox, Necitumumab, Nimotuzumab, Olaratumab, Oregovomab, Panitumumab, Pemtumomab, Pertuzumab, Ramucirumab, Rilotumumab, Robatumumab, Sibrotuzumab, Tacatuzumab tetraxetan, Tenatumomab, TGN1412, Ticilimumab, Tigatuzumab, TNX-650, Tremelimumab, Volociximab, Votumumab, Zalutumumab.

Table 1 gives a general overview of investigational and approved therapeutic antibodies. The present invention may be advantageously applied in the indicated treatment (use) of the respective therapeutic antibody (name).

TABLE 1
As an overview, the following table lists approved and investigational antibodies.

| Name | Type | Source | Target | Use |
|---|---|---|---|---|
| 3F8 | ? | mouse | GD2 | neuroblastoma |
| Abagovomab | mab | mouse | CA-125 | ovarian cancer |
| | | | (imitation) | |
| Adalimumab | mab | human | TNF-α | rheumatoid arthritis |
| | | | | etc. |
| Adecatumumab | mab | human | EpCAM | prostate and breast |
| | | | | cancer |
| Afutuzumab | mab | humanized | CD20 | lymphoma |
| Alacizumab pegol | F(ab′)2 | humanized | VEGFR2 | cancer |
| Alemtuzumab | mab | humanized | CD52 | CLL, CTCL |
| Anatumomab | Fab | mouse | TAG-72 | non-small cell lung |
| mafenatox | | | | carcinoma |
| Apolizumab | mab | humanized | HLA-DR ? | hematological cancers |
| Atlizumab | mab | humanized | IL-6 | rheumatoid arthritis |
| (=tocilizumab) | | | receptor | |
| Basiliximab | mab | chimeric | CD25 (α | prevention of organ |
| | | | chain of IL- | transplant rejections |
| | | | 2 receptor) | |
| Belimumab | mab | human | BAFF | non-Hodgkin |
| | | | | lymphoma etc. |
| Benralizumab | mab | humanized | CD125 | asthma |
| Bertilimumab | mab | human | CCL11 | severe allergic |
| | | | (eotaxin-1) | disorders |
| Bevacizumab | mab | humanized | VEGF-A | metastatic cancer |
| Bivatuzumab | mab | humanized | CD44 v6 | squamous cell |
| mertansine | | | | carcinoma |
| Blinatumomab | bispecific | mouse | CD19 | cancer |
| Brentuximab vedotin | mab | chimeric | CD30 | hematologic cancers |
| | | | (TNFRSF8) | |
| Cantuzumab | mab | humanized | mucin | colorectal cancer |
| mertansine | | | CanAg | etc. |
| Catumaxomab | 3funct | rat/mouse | EpCAM, | ovarian cancer, |
| | | hybrid | CD3 | malignant ascites, |
| | | | | gastric cancer |
| Cedelizumab | mab | humanized | CD4 | prevention of organ |
| | | | | transplant |
| | | | | rejections, treatment |
| | | | | of autoimmune |
| | | | | diseases |
| Cetuximab | mab | chimeric | EGFR | metastatic colorectal |
| | | | | cancer and head and |
| | | | | neck cancer |
| Citatuzumab bogatox | Fab | humanized | EpCAM | ovarian cancer and |
| | | | | other solid tumors |
| Cixutumumab | mab | human | IGF-1 | solid tumors |
| | | | receptor | |
| Clenoliximab | mab | chimeric | CD4 | rheumatoid arthritis |
| Clivatuzumab | mab | humanized | MUC1 | pancreatic cancer |
| tetraxetan | | | | |
| Conatumumab | mab | human | TRAIL-R2 | cancer |
| Dacetuzumab | mab | humanized | CD40 | hematologic cancers |
| Daclizumab | mab | humanized | CD25 (α | prevention of organ |
| | | | chain of IL- | transplant rejections |
| | | | 2 receptor) | |
| Denosumab | mab | human | RANKL | osteoporosis, bone |
| | | | | metastases etc. |
| Detumomab | mab | mouse | B- | lymphoma |
| | | | lymphoma | |
| | | | cell | |
| Ecromeximab | mab | chimeric | GD3 | malignant melanoma |
| | | | ganglioside | |
| Edrecolomab | mab | mouse | EpCAM | colorectal carcinoma |
| Efalizumab | mab | humanized | LFA-1 | psoriasis (blocks T- |
| | | | (CD11a) | cell migration) |
| Elotuzumab | mab | humanized | SLAMF7 | multiple myeloma |
| Epratuzumab | mab | humanized | CD22 | cancer, SLE |
| Erlizumab | F(ab′)2 | humanized | ITGB2 | heart attack, stroke, |
| | | | (CD18) | traumatic shock |
| Ertumaxomab | 3- | rat/mouse | HER2/neu, | breast cancer etc. |
| | functional | hybrid | CD3 | |
| Etaracizumab | mab | humanized | integrin | melanoma, prostate |
| | | | αvβ3 | cancer, ovarian |
| | | | | cancer etc. |
| Exbivirumab | mab | human | hepatitis B | hepatitis B |
| | | | surface | |
| | | | antigen | |
| Farletuzumab | mab | humanized | folate | ovarian cancer |
| | | | receptor 1 | |
| Felvizumab | mab | humanized | respiratory | respiratory syncytial |
| | | | syncytial | virus infection |
| | | | virus | |
| Figitumumab | mab | human | IGF-1 | adrenocortical |
| | | | receptor | carcinoma, non- |
| | | | | small cell lung |
| | | | | carcinoma etc. |
| Galiximab | mab | chimeric | CD80 | B-cell lymphoma |
| Gavilimomab | mab | mouse | CD147 | graft versus host |
| | | | (basigin) | disease |
| Gemtuzumab | mab | humanized | CD33 | acute myelogenous |
| ozogamicin | | | | leukemia |
| Glembatumumab | mab | human | GPNMB | melanoma, breast |
| vedotin | | | | cancer |
| Gomiliximab | mab | chimeric | CD23 (IgE | allergic asthma |
| | | | receptor) | |
| Ibritumomab tiuxetan | mab | mouse | CD20 | non-Hodgkin’s |
| | | | | lymphoma |
| Intetumumab | mab | human | CD51 | solid tumors |
| | | | | (prostate cancer, melanoma) |
| Inolimomab | mab | mouse | CD25 | graft versus host |
| | | | | disease |
| Inotuzumab | mab | humanized | CD22 | cancer |
| ozogamicin | | | | |
| Ipilimumab | mab | human | CD152 | melanoma |
| Iratumumab | mab | human | CD30 | Hodgkin’s lymphoma |
| | | | (TNFRSF8) | |
| Keliximab | mab | chimeric | CD4 | chronic asthma |
| Labetuzumab | mab | humanized | CEA | colorectal cancer |
| Lexatumumab | mab | human | TRAIL-R2 | cancer |
| Libivirumab | mab | human | hepatitis B | hepatitis B |
| | | | surface | |
| | | | antigen | |
| Lintuzumab | mab | humanized | CD33 | cancer |
| Lorvotuzumab | mab | humanized | CD56 | cancer |
| mertansine | | | | |
| Lucatumumab | mab | human | CD40 | multiple myeloma, |
| | | | | non-Hodgkin’s |
| | | | | lymphoma, |
| | | | | Hodgkin’s lymphoma |
| Lumiliximab | mab | chimeric | CD23 | chronic lymphocytic |
| | | | | leukemia |
| Mapatumumab | mab | human | TRAIL-R1 | cancer |
| Matuzumab | mab | humanized | EGFR | colorectal, lung and |
| | | | | stomach cancer |
| Milatuzumab | mab | humanized | CD74 | multiple myeloma |
| | | | | and other |
| | | | | hematological |
| | | | | malignancies |
| Mitumomab | mab | mouse | GD3 | small cell lung |
| | | | ganglioside | carcinoma |
| Nacolomab tafenatox | Fab | mouse | C242 | colorectal cancer |
| | | | antigen | |
| Naptumomab | Fab | mouse | 5T4 | non-small cell lung |
| estafenatox | | | | carcinoma, renal cell |
| | | | | carcinoma |
| Natalizumab | mab | humanized | integrin α4 | multiple sclerosis, |
| | | | | Crohn’s disease |
| Necitumumab | mab | human | EGFR | non-small cell lung |
| | | | | carcinoma |
| Nimotuzumab | mab | humanized | EGFR | squamous cell |
| | | | | carcinoma, head and neck |
| | | | | cancer, nasopharyngeal |
| | | | | cancer, glioma |
| Ocrelizumab | mab | humanized | CD20 | rheumatoid arthritis, |
| | | | | lupus erythematosus |
| | | | | etc. |
| Odulimomab | mab | mouse | LFA-1 | prevention of organ |
| | | | (CD11a) | transplant |
| | | | | rejections, |
| | | | | immunological |
| | | | | diseases |
| Ofatumumab | mab | human | CD20 | chronic lymphocytic |
| | | | | leukemia etc. |
| Olaratumab | mab | human | PDGF-R α | cancer |
| Oportuzumab | scFv | humanized | EpCAM | cancer |
| monatox | | | | |
| Oregovomab | mab | mouse | CA-125 | ovarian cancer |
| Panitumumab | mab | human | EGFR | colorectal cancer |
| Pemtumomab | ? | mouse | MUC1 | cancer |
| Pertuzumab | mab | humanized | HER2/neu | cancer |
| Priliximab | mab | chimeric | CD4 | Crohn’s disease, |
| | | | | multiple sclerosis |
| Pritumumab | mab | human | vimentin | brain cancer |
| Ramucirumab | mab | human | VEGFR2 | solid tumors |
| Rituximab | mab | chimeric | CD20 | lymphomas, |
| | | | | leukemias, some |
| | | | | autoimmune |
| | | | | disorders |
| Robatumumab | mab | human | IGF-1 | cancer |
| | | | receptor | |
| Rovelizumab | mab | humanized | CD11, CD18 | haemorrhagic shock |
| | | | | etc. |
| Ruplizumab | mab | humanized | CD154 | rheumatic diseases |
| | | | (CD40L) | |
| Sibrotuzumab | mab | humanized | FAP | cancer |
| Siplizumab | mab | humanized | CD2 | psoriasis, graft- |
| | | | | versus-host disease |
| | | | | (prevention) |
| Tacatuzumab | mab | humanized | alpha- | cancer |
| tetraxetan | | | fetoprotein | |
| Tadocizumab | Fab | humanized | integrin | percutaneous |
| | | | αIIbβ3 | coronary |
| | | | | intervention |
| Taplitumomab paptox | mab | mouse | CD19 | cancer[citation |
| | | | | needed] |
| Tenatumomab | mab | mouse | tenascin C | cancer |
| Teneliximab | mab | chimeric | CD40 | ? |
| TGN1412 | ? | humanized | CD28 | chronic lymphocytic |
| | | | | leukemia, |
| | | | | rheumatoid arthritis |
| Ticilimumab | mab | human | CTLA-4 | cancer |
| (=tremelimumab) | | | | |
| Tigatuzumab[ | mab | humanized | TRAIL-R2 | cancer |
| Tocilizumab | mab | humanized | IL-6 | rheumatoid arthritis |
| (=atlizumab) | | | receptor | |
| Toralizumab | mab | humanized | CD154 | rheumatoid arthritis, |
| | | | (CD40L) | lupus nephritis etc. |
| Tositumomab | ? | mouse | CD20 | follicular lymphoma |
| Trastuzumab | mab | humanized | HER2/neu | breast cancer |
| Tremelimumab | mab | human | CTLA-4 | cancer |
| Tucotuzumab | mab | humanized | EpCAM | cancer |
| celmoleukin | | | | |
| Vedolizumab | mab | humanized | integrin | Crohn’s disease, |
| | | | α4β7 | ulcerative colitis |
| Veltuzumab | mab | humanized | CD20 | non-Hodgkin’s |
| | | | | lymphoma |
| Volociximab | mab | chimeric | integrin | solid tumors |
| | | | α5β1 | |
| Votumumab | mab | human | tumor | colorectal tumors |
| | | | antigen | |
| | | | CTAA16.88 | |
| Zalutumumab | mab | human | EGFR | squamous cell |
| | | | | carcinoma of the |
| | | | | head and neck |
| Zanolimumab | mab | human | CD4 | rheumatoid arthritis, |
| | | | | psoriasis, T-cell |
| | | | | lymphoma |
| Zolimomab aritox | mab | mouse | CDS | systemic lupus |
| | | | | erythematosus, |
| | | | | graft-versus-host |
| | | | | disease |

Whether a target of a therapeutic binding molecule, such as a therapeutic antibody, is indeed found on exosomes may be tested, for example, as follows:

FACS analysis of exosomal surface proteins can be carried out after exosomes are coupled to latex beads as previously described (Thery, C., Zitvogel, L., & Amigorena, S. Nat. Rev. Immunol. 2, 569-579 (2002)). Exosomes (40 μg protein) are incubated in 30 μl PBS with 4 μm aldehyde/sulfate latex beads (3 μl of a 4% w/v suspension, Invitrogen) for 15 min., and again for 2 h after addition of 500 μl PBS at room temperature (RT) under constant agitation. The reaction is stopped by addition of 100 μl 1M glycine for 30 min. at RT with gentle agitation, followed by three washes in PBS/0.5% w/v bovine serum albumin (BSA). Antibody staining follows the protocols for antibody staining of cells in suspension by using an antibody directed against the surface marker to be detected. Detection of membrane-bound fluorescence is then performed using standard protocols for flow cytometry. 1×10⁵ cells per condition are washed once with PBS and thereafter incubated for 30 min at room temperature and protected from light with either primary antibody or isotype control (both 1:50).

With regard to the biogenesis of exosomes from lymphoma cells, the inventors here reveal, for the first time, a critical role of ABC transporter A3 in exosome release. ABCA3 is known to be an intracellular transporter indispensable for surfactant production from pneumocytes type 2. Structural similarity with other proteins of the group A of ABC transporters, as well as, functional data suggest a role for ABCA3 in lipid transport during biogenesis of specialized LROs, i.e. surfactant assembly in lamellar bodies. In murine ABCA3 knock-out models, the pneumocytes of heterozygote animals had fewer lamellar bodies and showed diminished incorporation of radiolabeled substrates into newly synthesized phospholipids, as well as a reduced content of phosphatidylcholine and phosphatidylglycerol in pulmonary surfactant. Sorting of membrane lipids is essential for MVB biogenesis and exosome release. Aggressive lymphoma cells exceed other hematological malignancies significantly in ABCA3 expression, consistent with their high levels of exosome secretion (cf. Table 2 below, Chapuy, B. et al. (2008), supra). The ABCA3 mediated mechanism described here links—for the first time—ABC transporter-mediated drug resistance and resistance against antibody-mediated lysis in a common pathway.

It is worth noting that ABCA3 expression has been described in a broad variety of tumor entities, so the inventors expect this resistance mechanism to be of relevance beyond leukemia and lymphomas (Chapuy, B. et al. (2008), supra; Steinbach, D. et al. (2006), supra, Hirschmann-Jax, C. et al. Proc. Natl. Acad. Sci. U.S.A 101, 14228-14233 (2004); Yasui, K. et al. Cancer Res. 64, 1403-1410 (2004); Yang, X., Liu, Y., Zong, Z., & Tian, D. Biomed. Pharmacother. 64, 58-62 (2010); Schimanski, S. et al. Horm. Metab Res. 42, 102-109 (2010); Song, J. H., Kim, S. H., Kim, H. J., Hwang, Y., & Kim, T. S. Int. J. Oncol. 32, 931-936 (2008)).

Importantly, inhibitors of exosome biogenesis or secretion augmented the cytolytic effects of anti-CD20 antibodies in the inventors' in vitro test systems. Several mechanisms have been described to interfere with cellular exosome secretion in different biological systems, ranging from agents perturbing multivesicular body (MVB) biogenesis such as rapamycin, to agents perturbing membrane cholesterol supply such as U18666A (Fader, C. M., Sanchez, D., Furlan, M., & Colombo, M. I. Traffic. 9, 230-250 (2008); Chalmin, F. et al. J. Clin. Invest 120, 457-471 (2010); Strauss, K. et al. J. Biol. Chem. (2010)). The inventors found all of these effects to enhance the cytolytic effect of rituximab.

Therefore, in a preferred embodiment, the exosome inhibiting agent is capable of perturbing multivesicular body (MVB) biogenesis. Whether the exosome inhibiting agent is capable of perturbing MVB biogenesis can be tested by imaging of subcellular morphology, e.g. by electronmicroscopy or subcellular fluorescence microscopy, using vesikel and/or exosome marker. For example, one may use the fluorescent phospholipid analog N-Rh-PE (Avanti Polar Lipids, Inc.). Briefly, an appropriate amount of the lipid, stored in chloroform/methanol (2:1) is dried under nitrogen and subsequently solubilized in absolute ethanol. This ethanolic solution is injected with a Hamilton syringe into serum-free (<1% v/v) culture media (e.g. RPMI) while vigorously vortexing. The mixture is then added to the cells, and they are incubated for 60 min at 4° C. After this incubation period, the medium is removed, and then the cells are extensively washed with cold PBS to remove excess unbound lipids. Labeled cells are cultured in complete culture medium (e.g. complete RPMI medium) for 3 h to allow internalized lipid to reach the MVBs. After this final incubation, cells were washed in PBS and immediately mounted on coverslips and analysed, e.g. by (confocal) subcellular fluorescence microscopy (cf. Fader et al. Traffic. 9, 230-250 (2008), incorporated herewith by reference).

Preferably, the MVB biogenesis in the cells incubated with the exosome formation inhibiting agent is at most 90% as compared to a corresponding cell not incubated with the exosome formation inhibiting agent, when cultured under otherwise comparable conditions. More preferably, the MVB biogenesis in the cells incubated with the exosome formation inhibiting agent is at most 80%, such as at most 75%, even more preferably at most 70%, e.g. at most 65%, or at most 60%, still even more preferably at most 55%, such as at most 50%, or at most 45%, and most preferably at most 40%, such as at most 35% or even at most 30%, at most 25%, or 20% as compared to a corresponding cell not incubated with the exosome formation inhibiting agent, when cultured under otherwise comparable conditions.

In one particular embodiment, the exosome inhibiting agent is an m-TOR inhibitor such as rapamycin or an analogue thereof. Examples of rapamycin analogues include (without limitation) those disclosed in EP 1 413 581, such as:

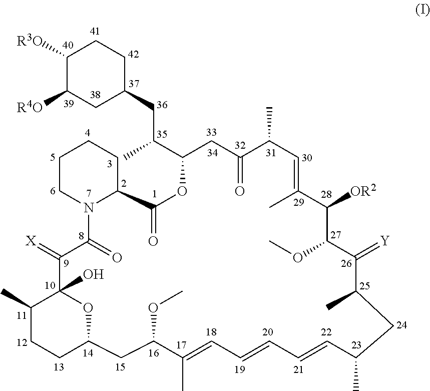

Wherein

X is (H,H) or O;

Y is (H,OH) or O;

R¹ and R² are independently selected from H, alkyl, thioalkyl, arylalkyl, hydroxyalkyl, dihydroxyalkyl, hydroxyalkylarylalkyl, dihydroxyalkylarylalkyl, alkoxyalkyl, acyloxyalkyl, aminoalkyl, alkylaminoalkyl, alkoxycarbonylaminoalkyl, acylaminoalkyl, arylsulfonamidoalkyl, allyl, dihydroxyalkylallyl, dioxolanylallyl, carbalkoxyalkyl, and (R³)₃Si where each R³ is independently selected from H, methyl, ethyl, isopropyl, t-butyl, and phenyl; wherein “alk-” or “alkyl” refers to C1-6 alkyl, branched or linear, preferably C1-3 alkyl, in which the carbon chain may be optionally interrupted by an ether (—O—) linkage; and

R⁴ is methyl or R⁴ and R¹ together form C2-6 alkylene;

provided that R¹ and R² are not both H; and

provided that where R¹ is carbalkoxyalkyl or (R³)₃Si, X and Y are not both O.

Preferred examples of these analogues are 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-V-yl]-rapamycin, (2′E,4′S)-40-O-(4′,5′-Dihydroxypent-2′-en-1′-yl)-rapamycin, 40-O-(2-Hydroxy)-ethoxycarbonylmethyl-rapamycin, 40-O-(2-Hydroxy)ethyl-rapamycin, 40-O-(3-Hydroxy)propyl-rapamycin, 40-O-(6-Hydroxy)hexyl-rapamycin, 40-O-[2-(2-Hydroxy)-ethoxy]ethyl-rapamycin, 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin, 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O—[2-(4′,5′-Dicarboethoxy-1′,2′,3′-triazol-V-yl)-ethyl]-rapamycin.

Further examples of potentially useful rapamycin analogues include, but are not limited to everolimus (40-O-(2-hydroxyethyl)-rapamycin; Grozinsky-Glasberg et al. Molecular and Cellular Endocrinology, 2010, 315(1-2), 87-94), PKI-587 (1-(4-{[4-(dimethylamino)piperidin-1-yl]carbonyl}phenyl)-3-[4-(4,6-dimorpholin-4-yl-1,3,5-triazin-2-yl)phenyl]urea; Venkatesan et al. Journal of Medicinal Chemistry, 2010, 53(6), 2636-2645), BAG956 (Weisberg et al. Blood, 2008, 111(7), 3723-34), temsirolimus (CCI779; Takayuki et al. Biochemical and Biophysical Research Communications, 2005, 331(1), 295-302), deforolimus (AP23573; ARIAD Pharmaceuticals, Cambridge, Mass., USA), SAR 943 (32-Deoxorapamycin; Sedrani et al., 1998, Transplant Proc 30:2192-2194), zotarolimus (ABT 578; Abbott Laboratories, Abbott Park, Ill., USA), AP20840 (ARIAD Pharmaceuticals, Cambridge, Mass., USA), AP21967 (Pollock et al., 2002, Nature Biotechnology 20, 729-733), and AP22565 (Pollock, R. et al., 2000, Proc. Natl. Acad. Sci. USA 97, 13221-13226). However, the rapamycin analogue must be capable of inhibiting exosome formation, e.g. by perturbing the ABCA3 function.

Therefore, in still another preferred embodiment, the exosome formation inhibiting agent is an inhibitor of a protein of the group A of ABC transporters (ABCA). The ATP-binding cassette (ABC) genes represent the largest family of transmembrane (TM) proteins. These proteins bind ATP and use the energy to drive the transport of various molecules across all cell membranes. Proteins are classified as ABC transporters based on the sequence and organization of their ATP-binding domain(s), also known as nucleotide-binding folds (NBFs). The NBFs contain characteristic motifs (Walker A and B), separated by approximately 90-120 amino acids, found in all ATP-binding proteins. ABC genes also contain an additional element, the signature (C) motif, located just upstream of the Walker B site. The functional protein typically contains two NBFs and two TM domains. The TM domains contain 6-11 membrane-spanning a-helices and provide the specificity for the substrate. The human ABCA subfamily currently comprises 12 full transporters that are further divided into two subgroups based on phylogenetic analysis and intron structure. The first group includes seven genes dispersed on six different chromosomes (ABCA1, ABCA2, ABCA3, ABCA4, ABCA7, ABCA12, ABCA13), whereas the second group contains five genes (ABCA5, ABCA6, ABCA8, ABCA9, ABCA10) arranged in a cluster on chromosome 17q24 (Micheal Dean, The Human ATP-Binding Cassette (ABC) Transporter Superfamily, Human Genetics Section, Laboratory of Genomic Diversity, National Cancer Institute-Frederick, 2002).

In a most preferred embodiment, the exosome formation inhibiting agent is an inhibitor of ABCA3.

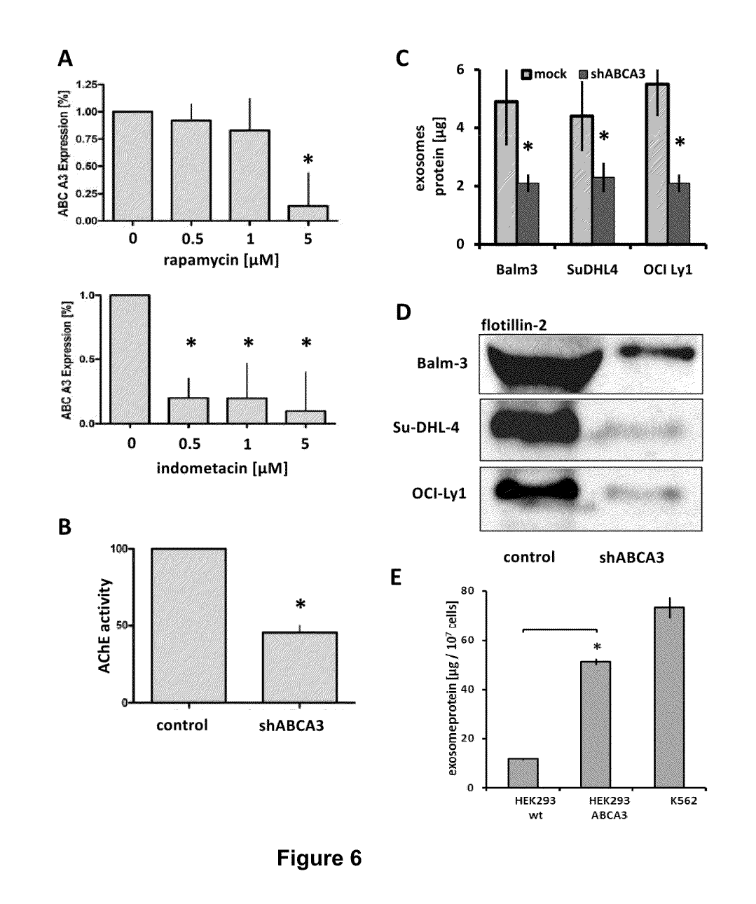

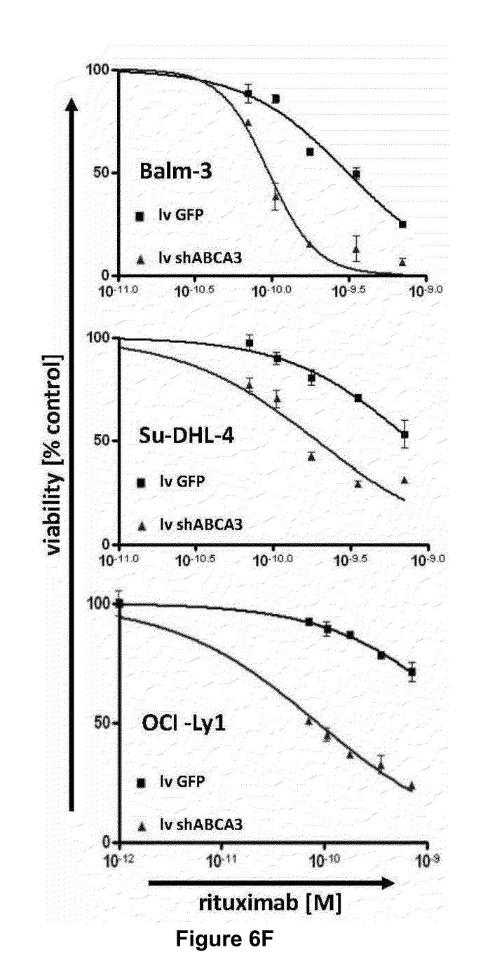

For indometacin, the central mechanism of inhibiting exosome release appears to be the downregulation of ABCA3 expression at the transcriptional level (FIG. 6A, and Song, J. H., Kim, S. H., Kim, H. J., Hwang, S. Y., & Kim, T. S. Int. J. Oncol. 32, 931-936 (2008)). Anti-cancer effects of indometacin have been consistently observed both clinically and in preclinical model systems, with a unifying mechanistic explanation for its efficacy not yet identified. Intriguingly, indometacin and other substances tested here would readily be available to be tested in combination with anti-CD20 antibodies in the clinical setting. Alternatively, the exosome formation inhibiting agent may be an agent which is capable of silencing ABCA3. “Silencing ABCA3” means that the expression level of ABCA3 in the cells incubated with the exosome formation inhibiting agent is at most 90% as compared to a corresponding cell not incubated with the exosome formation inhibiting agent, when cultured under otherwise comparable conditions. More preferably, the ABCA3 expression level in the cells incubated with the exosome formation inhibiting agent is at most 80%, such as at most 70%, even more preferably at most 60%, e.g. at most 50%, or at most 40%, still even more preferably at most 30%, such as at most 20%, or at most 10%, and most preferably at most 5%, such as at most 4% or even at most 3%, at most 2%, or at most 1% as compared to a corresponding cell not incubated with the exosome formation inhibiting agent, when cultured under otherwise comparable conditions.

Accordingly, in one preferred embodiment, the exosome formation inhibiting agent is indometacin, or an agent capable of silencing ABCA3. One example for an agent capable of silencing ABCA3 is an ABCA3-RNAi. RNAi techniques and methods of designing and producing RNAi are known in the art. Thus, in a more preferred embodiment, the exosome formation inhibiting agent is an ABCA3-RNAi, preferably wherein the ABCA3-RNAi comprises the sequence shown in SEQ ID NO: 1 and/or SEQ ID NO: 2.

In another preferred embodiment, the exosome inhibiting agent is capable of perturbing membrane cholesterol supply. Whether the exosome inhibiting agent is capable of perturbing membrane cholesterol supply may be tested by immunofluorescence and filipin staining according to s standard protocol, as described in Strauss et al. (J. Biol. Chem., 2010, 285(34), 26279-26288). For filipin labelling, cells are fixed with 4% paraformaldehyde before staining with filipin (Sigma) in PBS.

Preferably, the membrane cholesterol supply in the cells incubated with the exosome formation inhibiting agent is at most 90% as compared to a corresponding cell not incubated with the exosome formation inhibiting agent, when cultured under otherwise comparable conditions. More preferably, the membrane cholesterol supply in the cells incubated with the exosome formation inhibiting agent is at most 80%, such as at most 75%, even more preferably at most 70%, e.g. at most 65%, or at most 60%, still even more preferably at most 55%, such as at most 50%, or at most 45%, and most preferably at most 40%, such as at most 35% or even at most 30%, at most 25%, or 20% as compared to a corresponding cell not incubated with the exosome formation inhibiting agent, when cultured under otherwise comparable conditions.

One example of such an exosome formation inhibiting agent, which perturbs the membrane cholesterol supply is U1866A. Accordingly, in another preferred embodiment, the exosome inhibiting agent is U1866A.

In still another preferred embodiment, the exosome formation inhibiting agent is an inhibitor of phosphatidylinositol-3-kinase, such as wortmannin, 3-methyladenine, or LY294002, or wherein the exosome formation inhibiting agent is an inhibitor of ADAM-Metalloproteases, such as INCB3619; or wherein the exosome formation inhibitor is a calcium chelator, such as EGTA.

Further known phosphatidylinositol-3-kinase inhibitors (PI3-kinase inhibitors) are deforolimus, CCI-779, BAG956, PKI-587, and everolimus, as described and defined herein, as well as perifosine (KRX-0401), CAL101, BEZ235, SF1126, GDC-0941, BKM120, XL147, XL765, palomid 529, GSK615, ZSTK474, IC87114, TG100-115, CAL263, PI-103, SAR245408, SAR245409, and PWT33597. Wortmannin is a mycotoxin, which is produced in and may be obtained from Fusarium oxysporum, Fusarium avenaeceum, Fusarium sambucinum, or Penicillium funiculosum. In order to stabilize the Wortmannin molecule while not losing its therapeutic effect, numerous derivatives were synthesized from Wortmannin, such as PX-866, which has been shown to be a novel, potent, irreversible, inhibitor of PI-3 kinase with efficacy when delivered orally. PX-866 is currently in phase II clinical trials by Oncothyreon. Another wortmannin derivate is demethoxyviridin (cf. Yuan et al. Bioorg. Med. Chem. Lett. 19(15), 4223-4227 (2009), incorporated herewith by reference). LY294002 is a morpholine derivative of quercetin. LY294002 is a reversible inhibitor of PI3K whereas wortmannin acts irreversibly.

Keller et al. (Immunology Letters, 107, 102-108 (2006)) disclose that calcium flux stimulate exosome release and that inhibitors of ADAM-metalloproteases block exosome formation. Thus, the exosome formation inhibitor may be an inhibitor of ADAM-Metalloproteases, such as INCB3619; or a calcium chelator, such as EGTA.

Preferably, the at least one exosome formation inhibiting agent for use according to the invention, and said therapeutic binding molecule, e.g. the therapeutic antibody are formulated in a pharmaceutical composition.

The exosome formation inhibiting agent is administered before and/or concomitant to said therapeutic binding molecule, e.g. to the therapeutic antibody. The term “before” means that the exosome formation inhibiting agent is administered in a manner, that its effect on exosome formation acts prior to the action of the therapeutic binding molecule. For example, indomethacin is administered preferably before the therapeutic binding molecule, since it regulates ABCA3 on the transcriptional level, which therefore takes a certain time to take effect. For certain other exosome formation inhibiting agents, it is preferred that they are administered concomitant or essentially concomitant to the therapeutic binding molecule. The term “concomitant” or “essentially concomitant” means in this context, that the exosome formation inhibiting agent acts at the same time than the therapeutic binding molecule. For example, it is preferred that U18666 is administered concomitant with the therapeutic binding molecule, since the response to U18666 is more or less without any lag phase. The skilled person will know which exosome formation inhibiting agent may be administered before, concomitant, or before and concomitant to the therapeutic binding molecule. In a particular embodiment, correct administration of the exosome formation inhibiting agent and the therapeutic binding molecule may be ensured by convenient types of drug formulation, such as delayed release formulations, etc.

In one embodiment, the pharmaceutical composition according to the invention may comprise a pharmaceutically acceptable carrier, excipient and/or diluent. The choice of carrier may depend upon route of administration and concentration of the active agent(s) and the pharmaceutical composition may be in the form of a lyophilised composition or an aqueous solution. Generally, an appropriate amount of a pharmaceutically acceptable salt is used in the carrier to render the composition isotonic. Examples of the carrier include but are not limited to phosphate buffered saline, Ringer's solution, dextrose solution, water, emulsions, such as oil/water emulsions, various types of wetting agents, sterile solutions, etc. Preferably, acceptable excipients, carriers, or stabilisers are non-toxic at the dosages and concentrations employed, including buffers such as citrate, phosphate, and other organic acids; salt-forming counter-ions, e.g. sodium and potassium; low molecular weight (>10 amino acid residues) polypeptides; proteins, e.g. serum albumin, or gelatine; hydrophilic polymers, e.g. polyvinylpyrrolidone; amino acids such as histidine, glutamine, lysine, asparagine, arginine, or glycine; carbohydrates including glucose, mannose, or dextrins; monosaccharides; disaccharides; other sugars, e.g. sucrose, mannitol, trehalose or sorbitol; chelating agents, e.g. EDTA; non-ionic surfactants, e.g. Tween, Pluronics or polyethylene glycol; antioxidants including methionine, ascorbic acid and tocopherol; and/or preservatives, e.g. octadecyldimethylbenzyl ammonium chloride; hexamethonium chloride; benzalkonium chloride, benzethonium chloride; phenol, butyl or benzyl alcohol; alkyl parabens, e.g. methyl or propyl paraben; catechol; resorcinol; cyclohexanol; 3-pentanol; and m-cresol). Suitable carriers and their formulations are described in greater detail in Remington's Pharmaceutical Sciences, 17th ed., 1985, Mack Publishing Co.

The pharmaceutical composition may be administered to the subject at a suitable dose, i.e. about 1 ng/kg body weight to about 100 mg/kg body weight of a subject. In one embodiment of the present invention, the composition comprising a binding molecule as described herein comprises the binding molecule, e.g. the antibody (or a fragment or a derivative thereof) in an amount of about 10 ng/kg to about 5 mg/kg or to about 10 mg/kg per body weight.

Thus, in a preferred embodiment, the composition is administered to said subject at a dose of about 1 ng/kg body weight to about 100 mg/kg body weight of said subject, preferably at a dose of about 10 ng/kg to about 10 mg/kg, more preferably at a dose of about 10 ng/kg to about 5 mg/kg per body weight.

Administration of the compositions described and provided herein may be effected by different ways, e.g., enterally, orally (e.g., pill, tablet (buccal, sublingual, orally, liquid solution or suspension), rectally (e.g., suppository, enema), via injection (e.g., intravenously, subcutaneously, intramuscularly, intraperitoneally, intradermally) via inhalation (e.g., intrabronchially), topically, vaginally, epicutaneously, or intranasally. In a preferred embodiment, the composition is administered via injection, in particular intravenously, subcutaneously, intramuscularly, intraperitoneally, or intradermally; more particular wherein the composition is administered intravenously.

The dosage regimen will be determined by an attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions comprising a binding molecule, preferably an antibody molecule as described herein may be administered locally or systemically. Administration will preferably be intravenously but may also be an administration that is subcutaneously, intramuscularly, intraperitoneally, or even intracranially. The compositions comprising a binding molecule, preferably an antibody molecule or a fragment or derivative thereof as described herein may also be administered directly to the target site, e.g., by biolistic delivery to an internal or external target site or by catheter to a site in an artery or a vein.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, also doses below or above of the exemplary ranges described hereinabove are envisioned, especially considering the aforementioned factors.

In still another aspect, the invention relates to a kit of parts comprising at least one exosome formation inhibiting agent and a therapeutic binding molecule, e.g. a therapeutic antibody, suitable for the treatment of cancer, for use in the treatment of cancer which acquired or may acquire exosome mediated resistance against a therapeutic binding molecule, e.g. a therapeutic antibody, suitable for treating said cancer.

Likewise, the present invention also relates to a kit of parts, comprising at least one agent inhibiting exosome formation, and a therapeutic binding molecule, such as a therapeutic antibody, suitable for the treatment of a disease, for use in the treatment of said disease in a patient, wherein the therapeutic antibody is directed against an antigen which is present on exosomes isolated from said patient, with the provisio that if the antibody is Rituximab, the exosome formation inhibiting agent is not rapamycin, in particular if the disease is cancer.

In preferred embodiments, the exosome formation inhibiting agent and/or the therapeutic binding molecule are further defined as above.

In another aspect, the invention relates to a method for reducing exosome mediated tumor resistance against a therapeutic binding molecule, wherein the method comprises the steps of (i) administering an effective amount of at least one agent inhibiting exosome formation, and (ii) administering said therapeutic binding molecule, wherein step (i) is conducted before and/or concomitant to step (ii). Thereby, the terms have the meaning as described and defined above.

In still a further aspect, the invention also relates to a method of increasing the efficacy of a therapeutic binding molecule suitable in the treatment of a disease, wherein the method comprises the steps of (i) administering an effective amount of at least one agent inhibiting exosome formation, and (ii) administering said therapeutic binding molecule, wherein step (i) is conducted before and/or concomitant to step (ii), with the provisio that if the binding molecule is Rituximab, the exosome formation inhibiting agent is not rapamycin.

Again, the terms have the meaning as described and defined above.

Preferably the disease is cancer, more preferably a cancer which acquired or may acquire exosome mediated resistance against said therapeutic binding molecule, or wherein the disease is a proliferative autoimmune disease, such as psoriasis.

In one preferred embodiment, the binding molecule is selected from an antibody molecule, a polypeptide, peptide, peptidomimetic, or a small molecule having a molecular weight in the range of 250-800 Da, as described and defined above.

In another preferred embodiment, the exosome inhibiting agent is capable of perturbing multivesicular body (MVB) biogenesis, as described and defined above.

Preferably, the exosome inhibiting agent is rapamycin or an analogue thereof, as described and defined above.

In a particularly preferred embodiment, the exosome inhibiting agent is capable of perturbing membrane cholesterol supply. Thus, preferably, the exosome inhibiting agent is U1866A.

In another preferred embodiment, the exosome formation inhibiting agent is an inhibitor of a protein of the group A of ABC transporters, more preferably the exosome formation inhibiting agent is an inhibitor of ABCA3, such as indometacin, or an agent capable of silencing ABCA3, as described and defined above. A preferred example of an agent capable of silencing ABCA3 is an ABCA3-RNAi, preferably wherein the ABCA3-RNAi comprises the sequence shown in SEQ ID NO: 1 and/or SEQ ID NO: 2. In still another preferred embodiment, the exosome formation inhibiting agent is an inhibitor of phosphatidylinositol-3-kinase, such as wortmannin, 3-methyladenine, or LY294002, or wherein the exosome formation inhibiting agent is an inhibitor of ADAM-Metalloproteases, such as INCB3619; or wherein the exosome formation inhibitor is a calcium chelator, such as EGTA; as described and defined above.

Preferably, the binding molecule is an antibody molecule, selected from a polyclonal antibody, a monoclonal antibody, a recombinant full antibody (immunoglobulin), a F(ab)-fragment, a F(ab)2-fragment, a F(v)-fragment, a single-chain antibody, a chimeric antibody, a CDR-grafted antibody, a bivalent antibody-construct, a synthetic antibody, a cross-cloned antibody, a fully-human antibody, a humanized antibody, nanobodies, diabodies, and peptide aptamers, as described and defined above.

More preferably, the antibody is a cytolytic antibody, preferably wherein the antibody (i) directly induces apoptosis, (ii) mediates complement-dependent cytolysis, and/or (iii) mediates antibody-dependent cellular cytotoxicity.

In a particularly preferred embodiment, the therapeutic antibody is directed against CD20, CD40, CD19, CD23, EpCAM, or CD37, more preferably the therapeutic antibody is directed against CD20.

Accordingly, in a preferred embodiment, the therapeutic antibody is selected from the group of therapeutic antibodies consisting of Rituximab, Afutuzumab, Ibritumomab tiuxetan, Ofatumumab, Tositumomab, Veltuzumab, Blinatumomab, Dacetuzumab, Lucatumumab, Lumiliximab, Taplitumomab paptox, Adecatumumab, Catumaxomab, Edrecolomab, Oportuzumab monatox, Tucotuzumab celmoleukin, Efalizumab, Inolimomab, preferably wherein the therapeutic antibody is selected from Rituximab, Ibritumomab tiuxetan, Ofatumumab, Tositumomab, and Veltuzumab, most preferably wherein the therapeutic antibody is Rituximab.

Alternatively, the therapeutic binding molecule, e.g. the therapeutic antibody, may be directed against VEGFR2, GD2, CA-125, CD52, CEA, TAG-72, BAFF, VEGF-A, CD44, CD30, mucin CanAg, IGF1R, MUC1, TRAIL-R2, SLAMf7, CD22, HER2/neu, integrin αvβ3, folate receptor 1, TGF-β, GPNMB, CD51, CD152, CD33, CD56, EGFR, CD74, C242 antigen, 5T4, PDGF-Rα, VEGFR2, HGF, FAP, tenascin C, CTLA-4, IL-13, or integrin α5β1, TNF-R1, TNF-R2, IL-2R alpha (CD25), IL-2R beta (CD122), Fc epsilon R1, and CD11a.

Therapeutic antibodies which are directed against these surface markers, and which have been found or are tested for the treatment of cancer or a proliferative immune disease are known to the skilled person, and include, but are not limited to 3F8, Abagovomab, Alacizumab pegol, Alemtuzumab, Anatumomab, Apolizumab, Belimumab, Bevacizumab, Bivatuzumab, Brentuximab vedotin, Cantuzumab mertansine, Cetuximab, Citatuzumab bogatox, Cixutumumab, Clivatuzumab tetraxetan, Conatumumab, Detumomab, Ecromeximab, Elotuzumab, Epratuzumab, Ertumaxomab, Etaracizumab, Farletuzumab, Fresolimumab, Glembatumumab vedotin, Intetumumab, Inotuzumab ozogamicin, Ipilimumab, Iratumumab, Labetuzumab, Lexatumumab, Lintuzumab, Lorvotuzumab mertansine, Mapatumumab, Matuzumab, Milatuzumab, Mitumomab, Nacolomab tafenatox, Naptumomab estafenatox, Necitumumab, Nimotuzumab, Olaratumab, Oregovomab, Panitumumab, Pemtumomab, Pertuzumab, Ramucirumab, Rilotumumab, Robatumumab, Sibrotuzumab, Tacatuzumab tetraxetan, Tenatumomab, TGN1412, Ticilimumab, Tigatuzumab, TNX-650, Tremelimumab, Volociximab, Votumumab, Zalutumumab.

In a preferred embodiment, the cancer to be treated is selected from the group of cancers consisting of lymphoma, e.g. Non-Hodgkins lymphoma, Hodgkin's lymphoma, and follicular lymphoma; haematological cancers; chronic lymphocytic leukaemia (CLL); CTCL; lung cancer, e.g. non-small cell lung carcinoma; ovarian cancer; prostate cancer; and breast cancer. Preferably, the cancer is lymphoma, such as Non-Hodgkins lymphoma, Hodgkin's lymphoma, or follicular lymphoma; or haematological cancer; chronic lymphocytic leukaemia (CLL); CTCL; or lung cancer, e.g. non-small cell lung carcinoma. More preferably the cancer is lymphoma, such as Non-Hodgkins lymphoma, Hodgkin's lymphoma, or follicular lymphoma; or haematological cancer, even more preferably the cancer is lymphoma, such as Non-Hodgkins lymphoma, Hodgkin's lymphoma, or follicular lymphoma. Most preferably the cancer is lymphoma, e.g. Non-Hodgkins lymphoma. However, it is also contemplated that the method is useful in the treatment of further cancers, which involves the use of therapeutic binding molecules, such as therapeutic antibodies. Cancers which involve such a treatment are neuroblastoma; colorectal cancer; gastrointestinal cancer; squamous cell carcinoma; head and neck cancer; nasopharyngeal cancer; pancreatic cancer; and melanoma. Most preferably, the therapeutic binding molecule is the therapeutic antibody Rituximab and the disease is lymphoma, leukemia, or haematological cancer, more preferably a Non-Hodgkin-Lymphom.

It is further preferred that the at least one exosome formation inhibiting agent and said therapeutic antibody are formulated in a pharmaceutical composition, as described and defined above.

In the following, the present invention is illustrated by figures and examples which are not intended to limit the scope of the present invention.

### DESCRIPTION OF THE FIGURES

FIG. 1: Binding of Therapeutic Anti-Cd20 Monoclonal Antibody to Exosomes from B-Cell Lymphoma Cells.

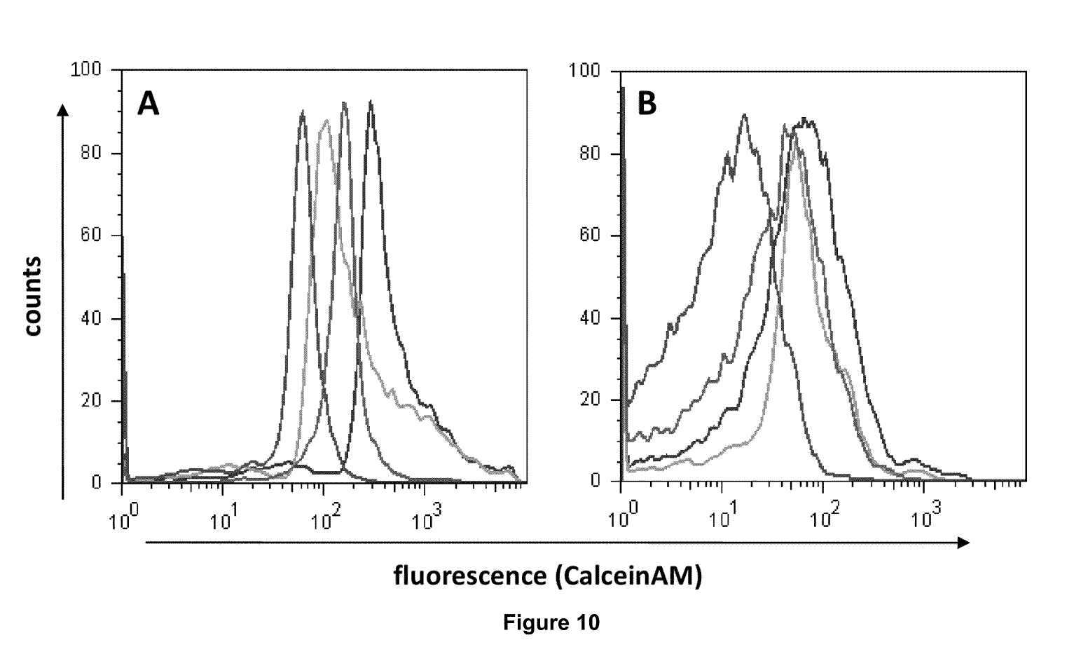

Differential ultracentrifugation of cell culture supernatants from B-cell lymphoma cell line Balm 3 yielded microvesicular structures with the typical size and morphology of exosomes at high purity (a, scale bar 100 nm). Purified and fixed exosomes were exposed to anti-CD20 antibody rituximab, and protein A-immunogold labelling revealed binding of rituximab to the exosomes (a, arrowheads), similar to the binding of rituximab on the cell surface (FIG. 7). Western blot confirmed binding of rituximab to exosomal protein preparations, detected with an anti-idiotype monoclonal antibody against rituximab (MB2A4), in parallel to the detection of the CD20 target protein and exosome markers alix and flotillin-2 (flot.-2, c). Exosomal binding of therapeutic monoclonal antibodies rituximab and GA101, as well as of CD20, flotillin-2, CD63 and CD9 were also documented by FACS with detection of binding to purified exosome-labelled beads (d, here Su-DHL-4). These findings were confirmed in several cell lines as well as in primary lymphoma exosome samples (FIGS. 9, 10). Besides CD20, complement regulatory proteins (CRPs) were detected on the cell surface of lymphoma cells and exosomes (d). Cells or exosomes coupled to beads were stained with fluorescence-labelled monoclonal antibodies, and analyzed by flow cytometry. Expression of CD20 (left columns), as well as of CRPs (right columns; CD46, CD55, CD59, isotype (isot.) control) differed between model cell lines. [From left to right: Su-DHL-4; cells: isot., CD59, CD55, CD46; exosomes: isot., CD46, CD55=CD59; Balm-3; cells: isot., CD55=CD59, CD46; exosomes: isot., CD46=CD59, CD55; OCI-Ly1; cells: isot., CD55, CD46, CD59; exosomes: isot. CD46, CD59=CD55]. However, levels of CRPs on the exosome surface were found uniformly high irrespective of the parental cell line.

FIG. 2: Absorption of Anti-Cd20 Antibody Rituximab and Consumption of Complement on Lymphoma-Derived Exosomes In Vitro and In Viva

Addition of exosomes from Su-DHL-4 cells to medium supplemented with rituximab at an initial concentration of 35 ng/100 μl for 1 h at 21° C. decreased the amount of soluble rituximab in a dose dependent manner (a). Soluble rituximab was measured by ELISA, and values were expressed as % of controls treated with non-exosome labelled beads only. Mean values of triplicates from a representative experiment are shown, with stars marking significant reduction compared to control (one-way ANOVA with Dunn's post test). As a control, soluble rituximab was not depleted by CD20 negative exosomes derived from K562 leukemia cells (FIG. 9). Rituximab binding to exosomes also occurred in vivo (b,c). Following ultracentrifugation preparation of exosomes from the plasma of patients at three hours after the end of infusion, rituximab binding to exosomes was detected with a specific antibody (MB2A4) by flow cytometry (b, further examples see FIG. 9). For quantification, rituximab was measured by ELISA, documenting approx. one third to half of plasma rituximab bound to exosomes, here with rituximab in the exosomal pellet represented in dark, and soluble rituximab from the supernatant in bright colour (c).

For the detection of complement fixation on the exosome surface, latex beads were coated with lymphoma exosomes, and exposed to rituximab in the presence of 20% human serum for 30 minutes. Formation of the terminal complement complex (TCC) was detected by indirect immunofluorescence with the anti-SC5b-9 primary monoclonal antibody WI3/15, a phycoerythrin (PE) labelled secondary antibody and measured by flow cytometry (e, upper row). As control for exosome labelling, the beads were stained for the exosome marker flotillin-2 (e, lower row). The finding was confirmed with exosomes from three representative patient samples taken three hours after exposure to rituximab (f). For an estimate of complement consumption, C3d levels were measured by ELISA using a polyclonal rabbit antibody (I3/15). Addition of beads labelled with exosomes (exo.) from Su-DHL-4 lymphoma cells in the presence of rituximab induced complement fixation and consumption in a dose-dependent pattern (d, right columns). PBS and unlabelled beads served as negative controls, zymosan as positive control for maximal complement fixation. Error bars indicate standard deviations of triplicates, a representative experiment of three replicates is shown. Asterices (*) denote results with statistical significance (one-way ANOVA with Dunn's post test).

FIG. 3: Rescue of Lymphoma Cells from Rituximab-Mediated CDC by Exosomes.

Exosomes (exo.) were added to OCI Ly-1 cells, followed by addition of rituximab at (ritux.) at EC50 (1 μg/ml). Viability was measured by MTT after an incubation at 37° C. for 1 h. Addition of exosomes, quantified by total protein, protected target cells from CDC dose-dependently, both with exosomes from an autologous source (OCI Ly-1, a), and with exosomes from allogeneic sources (Su-DHL-4, Karpas422, b). Representative examples of at least three replicates are shown, with standard deviations of triplicates indicated by errors bars, and significant differences (Student's two-sample t-test) compared to the samples with rituximab and without addition of exosomes marked by asterices (*). Vice versa, depletion of exosomes from the plasma of lymphoma patients increased cytolytic efficacy of rituximab. Patient plasma with or without depletion of exosomes were added to Su-DHL-4 target cells resulting in significantly higher cytotoxicity of rituximab in the absence of exosomes measured by MTT (FIG. 3c, one-way ANOVA with Dunn's post test, representative example of three experiments). Monitoring the cytolytic activity of patient plasma dilutions against autologous tumor cells ex vivo directly after rituximab infusion, again the exosome-depleted plasma revealed higher efficacy (FIG. 3d, Student's two-sample t-test).

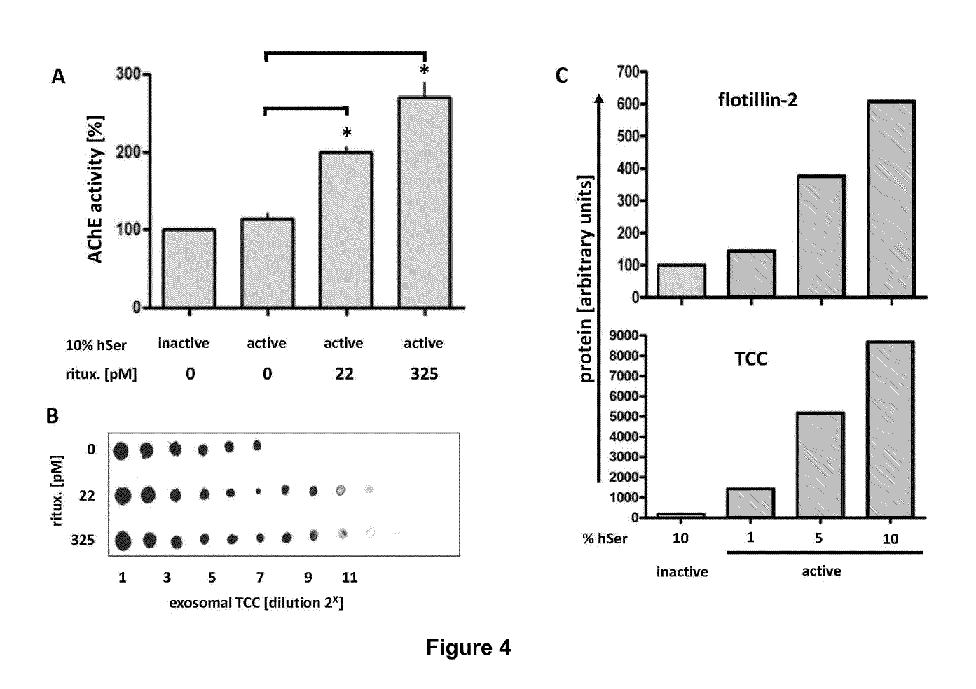

FIG. 4: Rituximab Stimulated Shedding of Exosomal Bound TCC from Lymphoma Cells.

5×10⁷ Su-DHL-4 lymphoma cells were exposed to sublytic concentrations of rituximab in the presence of complement (10% human serum, hSer), and exosomes were harvested after a 24 h incubation period. Yields of exosomes, quantified by AChE activity, increased with addition of rituximab (a, one-way ANOVA with Dunn's post test). Accordingly, rituximab induced an increase of exosomal bound terminal complement complex (TCC, w 10% active hSer), as measured by dot blot for W13/15 of exosome dilutions (b). Likewise, exosome release and fixation of TCC on exosomes increased with addition of active serum, quantified by densitrometry of dot blot (c, representative result of three experiments).

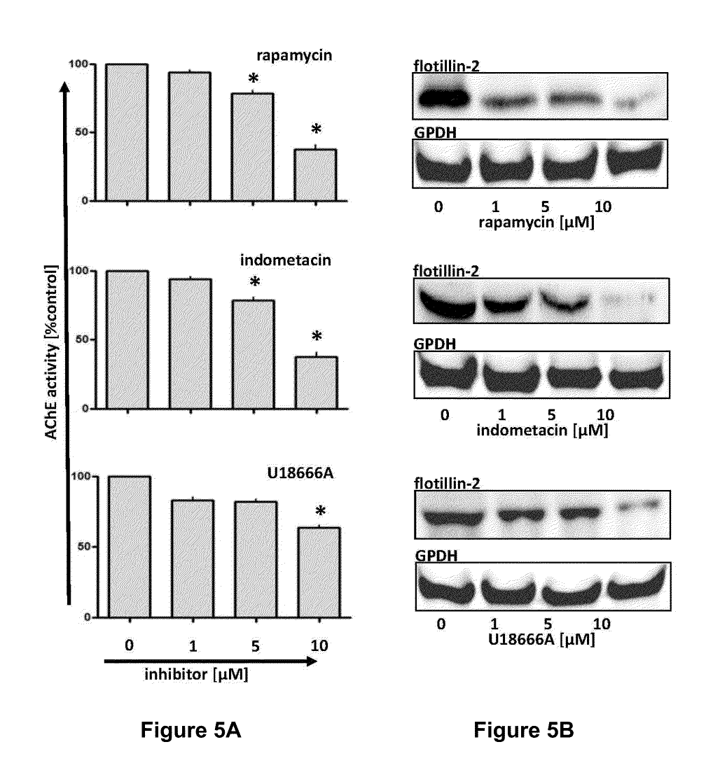

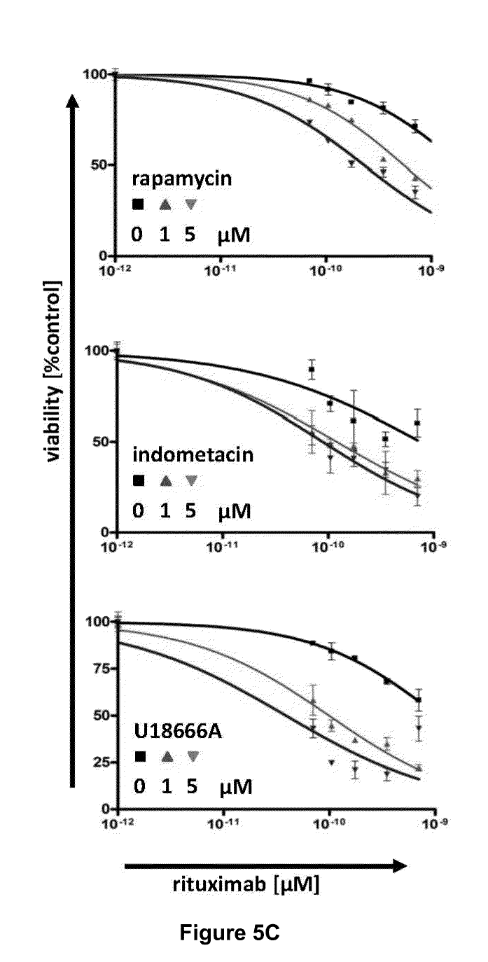

FIG. 5: Inhibition of Exosome Shedding and Enhanced CDC Susceptibility Induced by Rapamycin, Indometacin and U18666A.

5×10⁷⁰CI-Ly1 lymphoma cells were exposed to inhibitors of exosome synthesis or release at non-toxic dose ranges. Exosomes were harvested after a 24 h incubation period, and yields were measured by AChE activity (a) and detection of flotillin-2 by Western Blot (b). GPDH from corresponding exosome-producing cell samples are displayed as controls of protein content in treated cells (b). Significant differences to controls without inhibitors (one-way ANOVA with Dunn's post test) are marked with asterices, equivalent results were obtained for the cell lines Balm-3 and Su-DHL-4 (FIG. 10). Concomitant incubation of lymphoma cells with inhibitors and rituximab in the presence of 10% active human complement increased the cytolytic activity of the antibody in a dose-dependent manner (c).

FIG. 6: Role of ABCA3 for Exosome Release and Anti-Cd20 Mediated Complement Dependent Cytolysis.

Lymphoma cells were exposed to the inhibitors of exosome release for 48 h, and the expression of ABCA3 was measured by qRT/PCR, revealing a decrease of transporter expression by both rapamycin and indometacin (a, cell line Su-DHL-4, significant differences compared to controls tested by one-way ANOVA as marked by asterices). Accordingly, silencing gene expression by lentiviral anti-ABCA3 shRNA decreased exosome release, as compared to equal numbers of mock transfected controls. The effects were measured after incubation for 48 h by AChE activity (b, cell line Balm-3, significance tested by one-way ANOVA), whole exosomal protein (c, significance tested by Student's two-sample t-test), as well as flotillin-2 detection from exosome preparations in the cell culture supernatant (d). Addressing ABCA3 overexpression, 4×10⁷ cells of HEK293A, the stable ABCA3 overexpressing cell line HEK293A-ABCA3, as well as K562 cells were incubated for 17 h in 36 ml of exosome-free cell culture medium, and exosome yields measured as total exosomal protein. The exosome yields from the HEK 293A variant cell line significantly exceeded the yields from mock transduced HEK 293A cells (e, Students' t-test, p<0.05). RNAi mediated silencing of ABCA3 increased susceptibility of lymphoma cell lines to CDC (f). Lentiviral transfectants expressing either shRNA against ABCA3 (lv ABCA3, triangles [lower curves]) or green fluorescent protein (lv GFP, squares [upper curves]) were exposed to rituximab in the presence of complement, and viabilities were measured. Representative examples of three independent experiments performed in triplicates are shown, the differences between experimental (sh ABCA3) and controls (GFP) reached significance in all cell lines tested at rituximab concentrations above 10⁻¹⁰ M (Student's two-sample t-test).

FIG. 7: Morphology, Purity and Characterisation of Exosome Preparations from Aggressive B-Cell Lymphoma Cell Lines and Patient Samples.

Differential ultracentrifugation of cell culture supernatants from B-cell lymphoma cell lines yielded monomorphic microvesicular structures with the typical size and morphology of exosomes (A, Su-DHL-4, 100 nm), similar to the exosome preparations from the erythroleukemic cell line K562 (B, scale bar 100 nm). Such vesicles were also found accumulating in the intercellular space between lymphoma cells (arrow head in C, scale bar 1 μm). As a positive control to the binding of rituximab on exosomes (FIG. 1), binding of the antibody was also detected by protein A-immunogold staining on the surface of plasma protrusions (D, scale bar 200 nm).

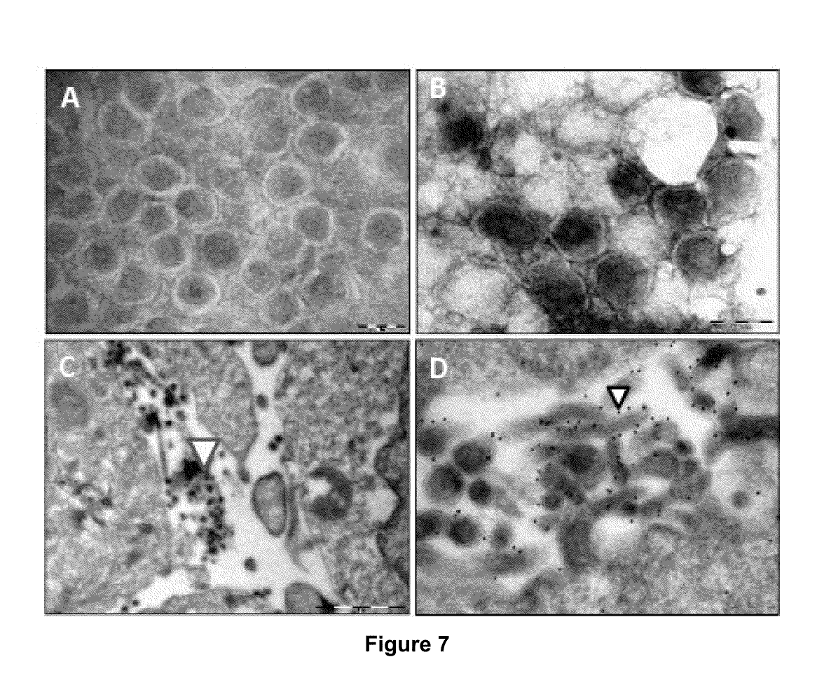

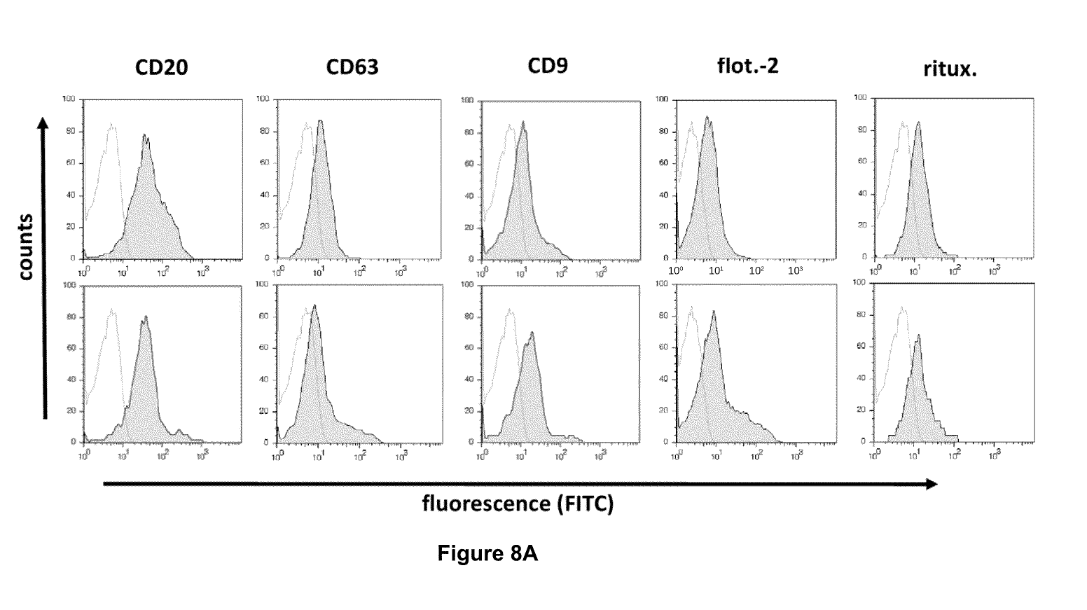

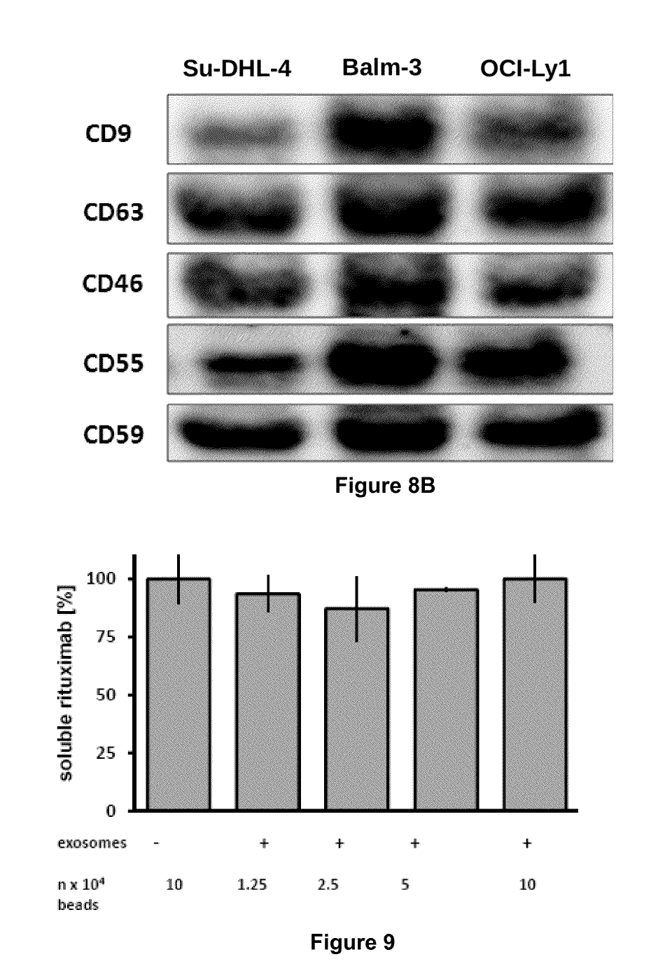

FIG. 8: Binding of Therapeutic Antibody Rituximab to Exosomes In Vivo.

Exosomes were prepared by ultracentrifugation from plasma samples of patients three hours after the end of rituximab infusion. Exosomes were coupled to beads, and binding of rituximab was detected by staining with a FITC-labelled antibody against rituximab (MB2A4). The samples originated from five patients with DLBCL (pts. 1, 2, 6, 7, 8), and one patient with immunocytoma (pt.5).

FIG. 9: No Absorption of Anti-CD20 Antibody Rituximab by CD20 Negative Exosomes Derived from K562 Cells.

Addition of exosomes from K562 cells to medium supplemented with rituximab at an initial concentration of 35 ng/100 μl for 1 h at 21° C. had no significant effect on the amount of soluble rituximab. Soluble rituximab was measured by ELISA, and values were expressed as % of controls treated with non-exosome labelled beads only. Mean values of triplicates from a representative experiment are shown.

FIG. 10: Resistance of Lymphoma Exosomes Against Rituximab-Initiated CDC.

Latex beads coated with exosomes of the cell line Balm-3 (A) and Su-DHL-4 (B) were labelled with CalceinAM and analyzed by flow cytometry. Fixation of complement by exposure to rituximab in the presence of 20% active human serum for 1 h at 37° C. (blue line) decreased CalceinAM fluorescence compared to exosomes after exposure to heat-inactived serum (negative control). Exposure to detergent agents (Fix&Perm®, SDS, as positive controls) lysed exosomes and induced maximal liberation of CalceinAM. Complement-mediated exosome destruction (−) induced low-level liberation of CalceinAM in both cell lines. [order of increasing graphs (not peaks!), from left to right; A: SDS, Fix&Perm®, exposure to rituximab, negative control; B: SDS, exposure to rituximab, Fix&Perm®, negative control]

FIG. 11: Inhibition of Exosome Shedding from Lymphoma Cells.

5×10⁷ lymphoma cells were exposed to inhibitors of exosome synthesis or release at non-toxic dose ranges. Exosomes were harvested after a 24 h incubation period, and yields were measured by AChE activity. Significant differences to controls without inhibitors (one-way ANOVA) are marked with asterices.

FIG. 12: Schematic Model of the Mechanisms Involved in Exosome-Mediated Protection of Lymphoma Cells from CDC Attack.

Following biogenesis in cytoplasmatic multivesicular bodies (MVB) facilitated by ABCA3, exosomes are released and shield lymphoma cells by absorption of rituximab to the CD20 positive exosomes (α), and by consumption of complement on the exosome surface (β).

### EXAMPLES

### Materials and Methods

Cells, Antibodies, Small Molecules, Plasmids and Vectors

The diffuse large B-cell lymphoma (DLBCL) cell lines Su-DHL-4 and Karpas 422 were obtained from a public depository (DSMZ, Braunschweig, Germany), the cell line Balm-3 (Lok, M. S. et al. Int J. Cancer. 24(5), 572-578 (1979)) was kindly provided by B. Glass and the cell OCI-Ly1 (Tweeddale, M. et al. Blood. 74(2), 572-578 (1989)) from the Ontario Cancer Instituts; the cell lines were propagated in RPMI 1640 supplemented with 25 mM HEPES, GlutaMAX I (Gibco-BRL), 1× penicillin/streptomycin (Sigma, Biochrom) and 10% heat-inactivated fetal calf serum (Gibco-BRL).

Approval for the collection and analysis of patient blood samples was obtained from the Institutional Review Board of the University Medicine Gottingen, and was conducted according to the Declaration of Helsinki. Each patient signed specific informed consent for the procedure. Cell viability was determined using 3-(4,5-dimethylthiazol-2-yl)-2,5-diphenyltetrazolium bromide (MU) assay as previously described (Denizot, F. & Lang, R. J. Immunol. Methods 89, 271-277 (1986)).

Briefly, cells were seeded in triplicates on 96-well culture plates at a density of 1×10⁵cells/well and were treated with the indicated concentrations of sorafenib and a DMSO control and then incubated at 37° C. After 24 hours, the culture volume of 100 μl was supplemented with MU in phosphate buffered saline (PBS) to achieve a final concentration of 0.5 mg/ml. After 4 hours incubation, suspension cells were spun, the supernatant discarded, and the pellet resuspended in 33% (v/v) dimethyl sulfoxide (DMSO) and 5% (v/v) formic acid (all from Sigma) dissolved in isopropanol. The light absorbance from formazan and background were measured at 540 nm and 655 nm on a Tecan SLT photometer (BioRad Mode1680 Microplate Reader). The inventors expressed the effect on viability as the ratio of values from treated versus untreated samples, i.e. specific viability is the ratio of absorbance with drug to absorbance of solvent control. IC50 was defined as the concentration of drug causing a 50% inhibition of cell growth as compared with untreated control and was determined using the curve fitting function (sigmoidal dose-response, variable slope) of Graph Pad Prism version 4.03 for Windows (GraphPad Software, San Diego Calif. USA, wwvv.graphpad.com).

The primary antibodies used in this study for the applications were as indicated: therapeutic anti-CD20 rituximab (type I) and GA101 (type II, both Roche), anti-rituximab idiotype (clone MB2A4, AbD serotec), anti-flotillin-2 (clone 29, BD-Pharmingen), anti-alix (clone 49/AIP1, BD-Pharmingen), anti-CD9 (clone M-L13, BD-Pharmingen), anti-CD63 (clone H5C6, BD-Pharmingen), anti-CD55 (clone IA10, BD-Pharmingen), anti-CD59 (clone p282, BD-Pharmingen), anti-CD46 (clone E4.3, BD-Pharmingen), anti-SC5b-9 monoclonal antibody W13/15 for the detection of terminal complement complex, (Wurzner, R. et al. Complement Inflamm. 8, 328-340 (1991)), anti-C3b/iC3b/C3dg monoclonal antibody 13/15 (Oppermann, M. et al. J. Immunol. Methods 133, 181-190 (1990)). Secondary antibodies against mouse or rabbit immunoglobulin were obtained from SantaCruz.

As small molecules were used at the concentrations indicated: mTOR inhibitor rapamycin, COX-2 inhibitor indometacin, cholesterol synthesis inhibitor U18666A (all from Sigma). As fluorescent dyes CalceinAM and PKH26 were applied (both from Molecular Probes). For silencing ABCA3 validated specific shRNA sequences (The RNAi Consortium, www.broadinstitute.org/rnai/trc:

forward 5′-CCGG(GCCCAGCTCATTGGGAAATTT)CTCGAG(AAATTTCCCAATGAGCTGGGC)TTTTTG-3′ (SEQ ID NO: 1);

reverse 5′-AATTCAAAAA(AAATTTCCCAATGAGCTGGGC)CTCGAG(GCCCAGCTCATTGGGAAATTT)-3′ (SEQ ID NO: 2))

were cloned into pLKO.1-eGF (Addgene), and lentiviral particles produced in HEK293T producer cell line with the plasmids pCMV-ΔR8.91 (containing gag, pol and rev genes) and pMD.G (VSV-G expressing plasmid) following standard protocols (Stewart, S. A. et al. RNA. 9(4), 493-501 (2003)).

Exosome Preparation and Quantification

Exosomes were prepared by differential centrifugation according to standard protocols (modified according to Valadi, H. et al. Nat. Cell Biol. 9, 654-659 (2007)). Following incubation of 5×10⁷ lymphoma cells for 48 h in complete exosome-free medium, cells and larger debris were removed by centrifugation for 10 min (10 min., 500 g, 4° C.). The supernatant was centrifuged again (20 min., 10000 g; 4° C.; Beckman L8-55 ultracentrifuge, rotor Ti32) to remove intermediate size particles. Subsequently the supernatant containing exosomes was filtered (0.22 μM Millex GP), and again centrifuged (240 min., 120 000 g, 4° C.; Beckman L8-55 ultracentrifuge, rotor Ti32) to obtain the exosome pellet, which was washed once in PBS, and finally resuspended in 50 μl PBS for further applications. Exosomes were quantified by measuring whole protein according to standard protocols (BioRad-DC-Protein-Assay), Western blot detecting flotillin-2 in comparison with whole cells or control exosome preparations, and acetyl-cholin-esterase (AChE) activity as previously described (Savina, A., Fader, C. M., Damiani, M. T., & Colombo, M. I. Traffic. 6, 131-143 (2005)).

LDS-PAGE, Flow Cytometry, Microscopy, Western Blot and PCR

For LDS-PAGE total cell lysates were prepared in Cellytic M (Sigma), supplemented with 1 mM Na₃VO₄, 10 mM Na₂MoO₄ and proteinase inhibitor cocktail (Sigma). 25 μg of protein were run on an LDS NuPage Novex 4-12% BisTris gradient Gel in accordance with the manufacturer's recommendations. Protein transfer was completed using 30V for 60 minutes on Hyperbond-C Extra (Amersham Biosciences), and blocked with 5% BSA (Sigma) in 0.1% Tris-buffered-saline. After washing, membranes were probed against the indicated antigens following the manufactures recommendation for the antibodies. Secondary HRP-conjugated antibodies against anti-rabbit or anti-mouse were purchased from Santa Cruz. For chemoluminescence detection standard ECL (Pierce) was used. For quantification of mRNA, qRT/PCR of hABCA3 and β-actin transcripts were performed in triplicates on a Taqman cycling machine (ABI Prism 7900HT Sequence Detection System, Applied Biosystems) following previously published protocols (Chapuy et al. 2008). Briefly, the SYBR green kit (Quiagen) was used according to the manufacturer's protocols, with 40 cycles of denaturation (15 seconds at 95° C.), annealing (45 seconds at 58° C.) and elongation (60 seconds at 72° C.) followed by a melting curve analysis. Subsequently, the threshold PCR cycle number (C7) was obtained when the increase in the fluorescence signal of the PCR product indicated exponential amplification (Livak & Schmittken 2001). This value was normalized to the threshold PCR cycle number obtained for β-actin mRNA from a parallel sample. The hABCA3 primer (us 5 ‘-TTCTTCACCTACATCCCCTAC-3′ (SEQ ID NO: 3); ds 5-CCTTTCGCCTCAAATTTCCC-3′ (SEQ ID NO: 4)) yielded an amplicon of 139 bp, the β-actin primer (us 5′-CACACTGTGCCCATCTACGA-3′ (SEQ ID NO: 5); ds 5′-TGAGGATCTTCATGAGCTAGTCAG-3′ (SEQ ID NO: 6)) an amplicon of 99 bp. A dilution series of eGFP-N1+ABCA3 (1×10⁻³−1×10⁻⁹M, Cheong, N. et al. J Biol Chem. 281(14), 9791-800 (2006)) was run in parallel with all reactions to allow comparison.

FACS analysis of exosomal surface proteins was carried out after exosomes were coupled to latex beads as previously described (Thery, C., Zitvogel, L., & Amigorena, S, Nat. Rev. Immunol. 2, 569-579 (2002)). Exosomes (40 μg protein) were incubated in 30 μl PBS with 4 μm aldehyde/sulfate latex beads (3 μl of a 4% w/v suspension, Invitrogen) for 15 min., and again for 2 h after addition of 500 μl PBS at room temperature (RT) under constant agitation. The reaction was stopped by addition of 100 μl 1M glycine for 30 min. at RT with gentle agitation, followed by three washes in PBS/0.5% w/v bovine serum albumin (BSA). Antibody staining followed the protocols for antibody staining of cells in suspension.

Detection of membrane-bound fluorescence were performed using standard protocols for flow cytometry. 1×10⁵ cells per condition were washed once with PBS and thereafter incubated for 30 min at room temperature and protected from light with either primary antibody or isotype control (both 1:50). Confocal microscopy and immunoelectron microscopy, were performed as follows.

For confocal microscopy, cells were fixed at RT using 3.7% paraformaldehyde for 20 minutes, with the subsequent quenching of any unspecific binding using 50 mM NH₄Cl for 15 minutes and permeabilization with 0.05% Triton X-100 in PBS for 15 minutes. Primary antibodies were diluted 1:100 in PBS for 1 hour. After washing twice with PBS and incubating with 10% goat serum, the primary antibodies were visualized using goat secondary antibodies at a dilution of 1:500 in PBS coupled to Cy3 (Dianova). All samples were mounted in Fluoromount (DAKO) and analyzed with the TCS-2 AOBS confocal laser scanning microscope (Leica) with a 63× inversion oil objective (Leica). The data were exported as TIFF files and arranged using Adobe PhotoShop® without further modification of the primary image. Immunoelectron microscopy was performed according to the Tokuyasu method. Cell and exosome samples were fixed with 2% paraformaldehyde and 0.1% glutaraldehyde in 0.1 M sodium phosphate [pH 7.4] at room temperature for 30 minutes, before the cells were postfixed with 4% paraformaldehyde and 0.1% glutaraldehyde on ice for 2 hours. After being washed twice with PBS—0.02% glycine, the cells were embedded in 10% gelatin, cooled on ice, and cut into small blocks. The blocks were infused with 2.3 M sucrose overnight and stored in liquid nitrogen. Ultrathin cryosections were cut from the frozen samples and labeled with primary antibodies detected with protein A conjugated to gold (PAG). In the case of monoclonal antibodies, a polyclonal rabbit anti-mouse bridging antibody (Sigma) was used prior to detection with PAG. Sections were contrasted with uranyl acetate methyl cellulose on ice for 10 minutes, embedded in the same solution, and examined with a Phillips CM120 electron microscope. In preparation of whole cell imaging, cells were fixed in 2% glutaraldehyde in 0.1 M PBS, pH 7.4, for 2 hours, postfixed in 1% OsO₄ for 1 hour, dehydrated in ethanol, and then embedded in Epon.

Cell and exosome lysates were prepared in Cellytic M (Sigma), and LDS-PAGE as well as western blot performed as described above. For quantification of mRNA, qRT/PCR of hABCA3 and β-actin transcripts were performed in triplicates on a Taqman cycling machine (ABI Prism 7900HT Sequence Detection System, Applied Biosystems) following previously published protocols (Chapuy, B. et al. Leukemia 22, 1576-1586 (2008), see above).

Complement Analysis and Rituximab Measurements

To detect and quantify rituximab in medium and patient serum samples, an ELISA using the anti-idiotypic monoclonal antibody MB2A4 was applied as previously described (Cragg, M. S. et al. Blood 104, 2540-2542 (2004)). Briefly, MB2A4 was coated at a concentration 5 μg/ml in coating buffer on 96-plates (Medisorb, Nunc), blocked, washed appropriately, and diluted rituximab-containing samples were added to the plate for 90 minutes. Following washes, the wells were incubated with anti-human Fc-horseradish peroxidase for 60 minutes, before further washing and addition of the 3,3′,5,5′-tetramethylbenzidine (TMB) substrate solution. TMB product was measured with a microplate reader at 450/540 nm. Background values from medium or matched serum samples before rituximab addition or infusion were subtracted, standard dilutions series of rituximab were run in parallel to allow rituximab quantification

To measure CDC activity, rituximab was added at the concentrations indicated to lymphoma cells (1×10⁵/100 μl) in complete medium supplemented with human serum (10%-20% v/v, as indicated) without prior heat inactivation (56° C. for 45 minutes). After 24 hours at 37° C., cell viability was determined in triplicates by M^{˜}7 staining as described before. The effect on viability was expressed as the ratio of values from treated versus untreated samples, i.e. specific viability=100× absorbance with antibody treatment/absorbance of untreated control. To detect complement decomposition of C3 in supernatant of exosome coupled to beads, monoclonal antibody 13/15 was coated onto 96-well Medisorp plates (NUNC, Medisorp, Thermo Fisher Scientific) blocked, and washed. Subsequently, appropriately diluted supernatant of exosome:bead complex samples were added to the plate for 60 minutes, washed, and incubated with rabbit anti-C3d for 60 min. After washing, finally anti-rabbit Fc conjugated to horseradish peroxidase (anti-rFc-HRP) was added for 60 minutes, before a further washing and addition of TMB substrate solution. Zymosan (Sigma) was used for maximal complement fixation as positive control, addition of beads without exosomes as negative control.

Statistical Evaluations

The indicated statistical tests were performed using GraphPad Prism version 4.03 for Windows (GraphPad Software, San Diego Calif. USA, www.graphpad.com), and differences with p<0.05 were considered significant. Error bars represent standard deviations of samples.

### Example 1

### Lymphoma-Derived Exosomes Bind Therapeutic Anti-CD20 Antibody

Applying ultracentrifugation techniques described for the isolation of exosomes from dendritic cells and erythropoietic progenitor cells (Thery, C., Zitvogel, L., & Amigorena, S, Nat. Rev. Immunol. 2, 569-579 (2002)), the inventors recovered monomorphic microvesicular structures of high purity with the typical size and morphology of exosomes in the supernatants from a series of aggressive B-cell lymphoma cell lines as well as from primary lymphoma cell preparations (FIG. 1a, FIG. 7, 8). The yields of exosomes were comparable to, or even surmounted, the amounts of exosomes harvested from cultures of K562, an erythroleukemic cell line widely used as model cell line for exosome release (cf. Table 1).

Table 2: Exosome Yields from Aggressive B-Cell Lymphoma Cell Lines and Lymphoma Samples.

5×10² cells of each cell line or tumor single cell suspension were incubated for 24 h in 36 ml of exosome-free cell culture medium. The supernatants were depleted of whole cells and debris by two consecutive steps of centrifugation (500 g 10 minutes and 10 000 g for 20 minutes), the exosomes were spun to pellet (100 000 g 120 minutes). Exosome yields are expressed as total exosomal protein. The exosome yields from aggressive lymphoma cell lines were equivalent to or higher as in the high level exosomes producing erythroleukemic cell line K562.

TABLE 2

| CELLS | PROTEIN [μg] |
|---|---|
| K562 | 101.0 ± 9.3 |
| Balm-3 | 98.4 ± 21.3 |
| Su-DHL-4 | 121.5 ± 39.0 |
| OCI-Ly1 | 205.2 ± 6.3 |
| pt. 1 | 137.7 ± 2.7 |
| pt. 2 | 141.3 ± 7.5 |
| pt. 3 | 141.6 ± 9.0 |

Such lymphoma-derived vesicles were positive for the exosome markers flotillin-2, alix, CD9, CD63 and the GPI-anchored complement regulatory proteins (CRPs) CD55 and CD59. Importantly, the exosomes also carried the B-cell plasma membrane protein CD20 (FIG. 1a-d, FIG. 9). The exosomal abundance of CD20 mirrored the expression of this protein in the parental cells, whereas the exosomal membrane levels of CD55, CD59 and CD46 were uniformly high on the exosomes from all cell lines, even when the parental cells showed only low level expression of the respective CRP (FIG. 1d). Lymphoma cell-derived exosomal CD20 bound the therapeutic anti-CD20 antibody rituximab (FIG. 1a-d), and thus effectively depleted the soluble antibody from antibody suspensions in vitro (FIG. 2 a, FIG. 9). In vivo, exosomes also bound the anti-CD20 antibody rituximab in humans who had received the antibody for therapeutic purposes (FIG. 2b, c; FIG. 11). Approximately half of all the plasma rituximab was found to be fixed to exosomes three hours after the end of the rituximab infusion (rituximab dose 375 mg/m², first course of R-CHOP immunochemotherapy, FIG. 2c). It is worth noting that these measurements were carried out with an ELISA using the monoclonal anti-idiotypic antibody MB2A4, which detects both soluble and membrane-bound rituximab (Cragg, M. S. et al. Blood 104, 2540-2542 (2004); Hampson, G. et al. J. Immunol. Methods (2010)). Thus the data provided herein are comparable to previous pharmacokinetic findings at initiation of rituximab therapy. However, the high fraction of rituximab bound to exosomes indicates that—at least at the beginning of monoclonal antibody therapy—significant proportions of rituximab in the serum are not in a soluble state and thus are not available for lymphoma cell attack.

### Example 2

### Lymphoma Exosomes Impede CDC

Rituximab exerts its cytocidal effects after CD20-ligation by initiating direct pro-apoptotic effects, complement-dependent cytotoxicity (CDC), and antibody-dependent cellular cytotoxicity (ADCC). In our cell line models the inventors found complement-mediated cytotoxicity to be the prevalent mechanism (FIG. 6c). Thus, binding of rituximab to the exosomes of cell lines led to fixation of complement on the exosome surface, detected by the terminal complement complex (TCC) with the antibody W13/15 on the exosomes, both in vitro and in patients in vivo (FIG. 2d,e). Moreover, exosomal complement fixation consumed plasma complement levels, measurable as the complement decay product C3d gradually increasing with exposure to increasing doses of exosomes (FIG. 2d). It is worth noting that lymphoma exosomes themselves were largely resistant against complement lysis, associated with their high expression levels of CRPs, and in congruence with previous data on exosomes derived from reticulocytes and antigen presenting cells (see above, and FIG. 11).

The consumption of both free antibody and complement raised our interest in the effects of exosomes as third party on rituximab-mediated lymphoma cells lysis. Therefore, the inventors exposed lymphoma cells to rituximab at EC50 in the presence of complement, adding exosomes at increasing concentrations. In these experiments, the inventors observed a dose-dependent protection of the lymphoma cell targets from antibody attack both from autologous exosomes, i.e. exosomes derived from the respective parental cell line, as well as from allogeneic exosomes derived from other cell lines (FIG. 3a,b). Importantly, the inventors found this protective effect of exosomes also to be effective in lymphoma patients treated with rituximab. Depletion of exosomes from the patient plasma not only increased the cytolytic efficacy against cell line targets (FIG. 3c), but also the cytolytic activity of the rituximab-containing plasma after infusion against the patient's autologous tumor cells was significantly enhanced by depletion of exosomes (FIG. 3d).

Sublytic complement attack induces a variety of biological effects in the target cells. Therefore the inventors looked for the effects of complement fixation by low levels of rituximab, and discovered a significant increase in exosome release from lymphoma under attack (FIG. 4a). Increased exosome release from the lymphoma cells led to a concomitant increased shedding of terminal complement complex, depending on the amounts of both rituximab and of complement in the medium (FIG. 4b,c). Thus, exosomes provide shielding of target cells against antibody-mediated complement attack both as a constitutive property of lymphoma cells, and as an adaptive immune evasive response.

### Example 3

### Enhanced CDC Efficacy by Inhibition of Exosome Release and Silencing of ABC Transporter A3

Several mechanisms have been described to interfere with cellular exosome secretion in different biological systems, ranging from agents perturbing multivesicular body (MVB) biogenesis such as rapamycin, to agents perturbing membrane cholesterol supply such as U18666A. The inventors applied such substances to lymphoma cell lines, and confirmed significant inhibitions of exosome release occurring at non-toxic concentrations of the drugs, regardless of the respective mechanisms (FIG. 5a, FIG. 11). Importantly, diminished exosome release was associated with increased lytic efficacy of rituximab in CDC experiments (FIG. 5c). In addition to the known inhibitors of exosome release such as rapamycin, and U18666A the inventors also discovered that the cyclooxygenase type-2 inhibitor indometacin impeded exosome release, and that indometacin sensitized the lymphoma cells to rituximab-mediated CDC (FIG. 5c). Indeed, indometacin strongly diminished ABCA3 expression in our B-cell lymphoma cell lines, which show high levels of ABCA3 expression (FIG. 6a,b). Suppression of ABCA3 expression by rapamycin and indometacin efficiently suppressed exosome release from the lymphoma cells (FIGS. 5 and 6b). In agreement with the effects of pharmacological blockades, silencing of ABCA3 by lentiviral RNAi reduced exosome release from lymphoma cells in the cell line models Balm-3, Su-DHL-4 and OCI-Ly1 by approximately 50% (FIG. 6 a-d). Concordantly, genetic silencing of ABCA3 also significantly increased the susceptibility of the lymphoma cells to CDC-mediated lysis (FIG. 6e). Vice versa, overexpression of ABCA3 alone was also sufficient to enhance exosome release from HEK 293 cells (FIG. 6e). Together, these data show that ABCA3 positively modulates exosome release from B-cell lymphoma cells. This modulation is critical for the antibody susceptibility of lymphoma cells and offers a target for pharmacological intervention.

### LIST OF REFERENCES

EP 1 413 581
WO 2010/056337
Bard, M. P. et al. Proteomic analysis of exosomes isolated from human malignant pleural effusions. Am. J. Respir. Cell Mol. Biol. 31, 114-121 (2004).
Buschow, S. I. et al. MHC class II-associated proteins in B-cell exosomes and potential functional implications for exosome biogenesis. Immunology and Cell Biology 88, 851-856 (2010).
Chalmin, F. et al. Membrane-associated Hsp72 from tumor-derived exosomes mediates STAT3-dependent immunosuppressive function of mouse and human myeloid-derived suppressor cells. J. Clin. Invest 120, 457-471 (2010).
Chapuy, B. et al. ABC transporter A3 facilitates lysosomal sequestration of imatinib and modulates susceptibility of chronic myeloid leukemia cell lines to this drug. Haematologica 94, 1528-1536 (2009).
Chapuy, B. et al. Intracellular ABC transporter A3 confers multidrug resistance in leukemia cells by lysosomal drug sequestration. Leukemia 22, 1576-1586 (2008).
Cheong, N. et al. Functional and trafficking defects in ATP binding cassette A3 mutants associated with respiratory distress syndrome. J Biol. Chem. 281(14), 9791-800 (2006).
Cragg, M. S. et al. A new anti-idiotype antibody capable of binding rituximab on the surface of lymphoma cells. Blood 104, 2540-2542 (2004).
Dean, M. The Human ATP-Binding Cassette (ABC) Transporter Superfamily, Human Genetics Section, Laboratory of Genomic Diversity, National Cancer Institute-Frederick, (2002).
Denizot, F. & Lang, R. Rapid colorimetric assay for cell growth and survival. Modifications to the tetrazolium dye procedure giving improved sensitivity and reliability. J. Immunol. Methods 89, 271-277 (1986).
Efferth, T. et al. Expression profiling of ATP-binding cassette transporters in childhood T-cell acute lymphoblastic leukemia. Mol. Cancer Ther. 5, 1986-1994 (2006).
Escudier, B. et al. Vaccination of metastatic melanoma patients with autologous dendritic cell (DC) derived-exosomes: results of thefirst phase I clinical trial. J. Transl. Med. 3, 10 (2005).
Fader, C. M., Sanchez, D., Furlan, M., & Colombo, M. I. Induction of autophagy promotes fusion of multivesicular bodies with autophagic vacuoles in k562 cells. Traffic. 9, 230-250 (2008).
Gisselbrecht, C. Use of rituximab in diffuse large B-cell lymphoma in the salvage setting. Br. J. Haematol. 143, 607-621 (2008).
Grozinsky-Glasberg et al. Molecular and Cellular Endocrinology, 315(1-2), 87-94 (2010).
Hampson, G. et al. Validation of an ELISA for the determination of rituximab pharmacokinetics in clinical trials subjects. J. Immunol. Methods (2010).
Hirschmann-Jax, C. et al. A distinct “side population” of cells with high drug efflux capacity in human tumor cells. Proc. Natl. Acad. Sci. U.S.A 101, 14228-14233 (2004).
Lok, M. S. et al. Establishment and characterization of human B-lymphocytic lymphoma cell lines (BALM-3, -4 and -5); intraclonal variation in the B-cell differentiation stage. Int J Cancer. 24(5), 572-578 (1979).
Oppermann, M. et al. Quantitation of components of the alternative pathway of complement (APC) by enzyme-linked immunosorbent assays. J. Immunol. Methods 133, 181-190 (1990).
Pollock, R. et al., Proc. Natl. Acad. Sci. USA 97, 13221-13226 (2000).
Pollock et al., Nature Biotechnology 20, 729-733 (2002).
Savina, A., Fader, C. M., Damiani, M. T., & Colombo, M. I. Rab11 promotes docking and fusion of multivesicular bodies in a calcium-dependent manner. Traffic. 6, 131-143 (2005).
Schimanski, S. et al. Expression of the lipid transporters ABCA3 and ABCA1 is diminished in human breast cancer tissue. Horm. Metab Res. 42, 102-109 (2010).
Sedrani et al., Transplant Proc. 30: 2192-2194 (1998).
Song, J. H., Kim, S. H., Kim, H. J., Hwang, S. Y., & Kim, T. S. Alleviation of the drug-resistant phenotype in idarubicin and cytosine arabinoside double-resistant acute myeloid leukemia cells by indomethacin. Int. J. Oncol. 32, 931-936 (2008).
Steinbach, D. et al. ABCA3 as a possible cause of drug resistance in childhood acute myeloid leukemia. Clin. Cancer Res. 12, 4357-4363 (2006).
Stewart, S. A. et al. Lentivirus-delivered stable gene silencing by RNAi in primary cells. RNA. 9(4), 493-501 (2003)
Strauss, K. et al. Exosome secretion ameliorates lysosomal storage of cholesterol in Niemann-Pick type C disease. J. Biol. Chem. 285(34), 26279-26288 (2010).
Thery, C., Ostrowski, M., & Segura, E. Membrane vesicles as conveyors of immune responses. Nat. Rev. Immunol. 9, 581-593 (2009).
Thery, C., Zitvogel, L., & Amigorena, S. Exosomes: composition, biogenesis and function. Nat. Rev. Immunol. 2, 569-579 (2002).
Trajkovic, K., Dhaunchak, A. S., Goncalves, J. T., Wenzel, D., Schneider, A., Bunt, G., Nave, K. A., and Simmons, M. Neuron to glia signaling triggers myelin membrane exocytosis from endosomal storage sites J. Cell Biol. 172, 937-948 (2006)
Tweeddale, M. et al. Production of growth factors by malignant lymphoma cell lines. Blood. 74(2), 572-578 (1989).
Valadi, H. et al. Exosome-mediated transfer of mRNAs and microRNAs is a novel mechanism of genetic exchange between cells. Nat. Cell Biol. 9, 654-659 (2007).
Venkatesan et al. Journal of Medicinal Chemistry, 53(6), 2636-2645 (2010).
Wanner, K. et al. Mammalian target of rapamycin inhibition induces cell cycle arrest in diffuse large B cell lymphoma (DLBCL) cells and sensitises DLBCL cells to rituximab. Br. J. Haematol. 134, 475-484 (2006).
Weisberg et al. Blood, 111(7), 3723-34 (2008).
Wurzner, R. et al. Inhibition of terminal complement complex formation and cell lysis by monoclonal antibodies. Complement Inflamm. 8, 328-340 (1991).
Yang, X., Liu, Y., Zong, Z., & Tian, D. The Rho kinase inhibitor fasudil inhibits the migratory behaviour of 95-D lung carcinoma cells. Biomed. Pharmacother. 64, 58-62 (2010).
Yasui, K. et al. Alteration in copy numbers of genes as a mechanism for acquired drug resistance. Cancer Res. 64, 1403-1410 (2004).
Yuan et al. Bioorg. Med. Chem. Lett. 19(15), 4223-4227 (2009).
Zitvogel, L. et al. Eradication of established murine tumors using a novel cell-free vaccine: dendritic cell-derived exosomes. Nat. Med. 4, 594-600 (1998).

What is claimed is:

1. A method for reducing exosome mediated tumor resistance against a therapeutic binding molecule, comprising the steps of
(i) administering an effective amount of at least one agent inhibiting exosome formation, and
(ii) administering said therapeutic binding molecule,
wherein step (i) is conducted before or concomitant to step (ii).

2. The method of claim 1, wherein the therapeutic binding molecule is an antibody molecule, a polypeptide, peptide, peptidomimetic, or a small molecule having a molecular weight in the range of 250-800 Da.

3. The method of claim 1, wherein the at least one agent inhibiting exosome formation is capable of perturbing multivesicular body (MVB) biogenesis.

4. The method of claim 1, wherein the at least one agent inhibiting exosome formation is capable of perturbing membrane cholesterol supply.

5. The method of claim 1, wherein the at least one agent inhibiting exosome formation is an inhibitor of a protein of the group A of ABC transporters.

6. The method of claim 1, wherein the at least one agent inhibiting exosome formation is an inhibitor of phosphatidylinositol-3-kinase, or an inhibitor of ADAM-metalloproteases, or a calcium chelator.

7. The method of claim 1, wherein the therapeutic binding molecule is directed against CD20, CD40, CD19, CD23, EpCAM, or CD37.

8. The method of claim 2, wherein the at least one agent inhibiting exosome formation and said therapeutic antibody are formulated in a pharmaceutical composition.

9. A method of increasing the efficacy of a therapeutic binding molecule suitable in the treatment of a disease, comprising the steps of
(i) administering an effective amount of at least one agent inhibiting exosome formation, and
(ii) administering said therapeutic binding molecule,
wherein step (i) is conducted before and/or concomitant to step (ii), and
wherein, if the binding molecule is Rituximab, the exosome formation inhibiting agent is not rapamycin.

10. The method of claim 9, wherein the disease is a disease that acquired or may acquire exosome mediated resistance against said therapeutic binding molecule.

11. The method of claim 10, wherein the disease is cancer.

12. The method of claim 11, wherein the cancer is selected from the group of cancers consisting of lymphoma, haematological cancers, chronic lymphocytic leukaemia (CLL), CTCL, lung cancer, ovarian cancer, prostate cancer, and breast cancer.

13. The method of claim 12, wherein the lymphoma is Non-Hodgkins lymphoma, Hodgkin's lymphoma, or follicular lymphoma.

14. The method of claim 12, wherein the lymphoma is Non-Hodgkins lymphoma.

15. The method of claim 12, wherein the lung cancer is non-small cell lung carcinoma.

16. The method of claim 12, wherein the disease is lymphoma or leukemia.

17. The method of claim 12, wherein the lymphoma is Non-Hodgkins lymphoma.

18. The method of claim 10, wherein the disease is a proliferative autoimmune disease.

19. The method of claim 18, wherein the proliferative autoimmune disease is psoriasis.

20. The method of claim 9, wherein the therapeutic binding molecule is an antibody molecule, a polypeptide, peptide, peptidomimetic, or a small molecule having a molecular weight in the range of 250-800 Da.

21. The method of claim 9, wherein the at least one agent inhibiting exosome formation is capable of perturbing multivesicular body (MVB) biogenesis.

22. The method of claim 21, wherein the at least one agent inhibiting exosome formation is rapamycin or an analogue thereof.

23. The method of claim 9, wherein the at least one agent inhibiting exosome formation is capable of perturbing membrane cholesterol supply.

24. The method of claim 23, wherein the at least one agent inhibiting exosome formation is U1866A.

25. The method of claim 9, wherein the at least one agent inhibiting exosome formation is an inhibitor of a protein of the group A of ABC transporters.

26. The method of claim 25, wherein the at least one agent inhibiting exosome formation is an inhibitor of ABCA3.

27. The method of claim 26, wherein the at least one agent inhibiting exosome formation is indometacin, or an agent capable of silencing ABCA3.

28. The method of claim 27, wherein the at least one agent inhibiting exosome formation is an ABCA3-RNAi.

29. The method of claim 28, wherein the ABCA3-RNAi comprises the sequence of SEQ ID NO: 1 and/or SEQ ID NO: 2.

30. The method of claim 9, wherein the at least one agent inhibiting exosome formation is an inhibitor of phosphatidylinositol-3-kinase, or an inhibitor of ADAM-metalloproteases, or a calcium chelator.

31. The method of claim 30, wherein the at least one agent inhibiting exosome formation is wortmannin, 3-methyladenine, demethoxyviridin, or LY294002.

32. The method of claim 30, wherein the at least one agent inhibiting exosome formation is INCB3619.

33. The method of claim 30, wherein the at least one agent inhibiting exosome formation is EGTA.

34. The method of claim 9, wherein the therapeutic binding molecule is directed against CD20, CD40, CD19, CD23, EpCAM, or CD37.

35. The method of claim 34, wherein the therapeutic binding molecule is directed against CD20.

36. The method of claim 34, wherein the therapeutic binding molecule is an antibody molecule, selected from the group consisting of a polyclonal antibody, a monoclonal antibody, a recombinant full antibody (immunoglobulin), a F(ab)-fragment, a F(ab)2-fragment, a F(v)-fragment, a single-chain antibody, a chimeric antibody, a CDR-grafted antibody, a bivalent antibody-construct, a synthetic antibody, a cross-cloned antibody, a fully-human antibody, a humanized antibody, nanobodies, diabodies, and peptide aptamers.

37. The method of claim 36, wherein the antibody is a cytolytic antibody.

38. The method of claim 36, wherein the antibody (i) directly induces apoptosis, (ii) mediates complement-dependent cytolysis, and/or (iii) mediates antibody-dependent cellular cytotoxicity.

39. The method of claim 36, wherein the antibody is selected from the group of therapeutic antibodies consisting of Rituximab, Afutuzumab, Ibritumomab tiuxetan, Ofatumumab, Tositumomab, Veltuzumab, Blinatumomab, Dacetuzumab, Lucatumumab, Lumiliximab, Taplitumomab paptox, Adecatumumab, Catumaxomab, Edrecolomab, Oportuzumab monatox, Tucotuzumab celmoleukin, Efalizumab, and Inolimomab.

40. The method of claim 36, wherein the antibody is Rituximab, Ibritumomab tiuxetan, Ofatumumab, Tositumomab, or Veltuzumab.

41. The method of claim 36, wherein the antibody is Rituximab.

42. The method of claim 36, wherein the at least one agent inhibiting exosome formation and said antibody are formulated in a pharmaceutical composition.


SEQUENCE LISTING (6 sequences)

SEQ ID NO: 1 - DNA, 58 bp, Artificial Sequence
  synthetic shRNA forward
ccgggcccagctcattgggaaatttctcgagaaatttcccaatgagctgggctttttg

SEQ ID NO: 2 - DNA, 58 bp, Artificial Sequence
  synthetic shRNA reverse
aattcaaaaaaaatttcccaatgagctgggcctcgaggcccagctcattgggaaattt

SEQ ID NO: 3 - DNA, 21 bp, Artificial Sequence
  synthetic primer hABCA3 us
ttcttcacctacatcccctac

SEQ ID NO: 4 - DNA, 20 bp, Artificial Sequence
  synthetic primer hABCA3 ds
cctttcgcctcaaatttccc

SEQ ID NO: 5 - DNA, 20 bp, Artificial Sequence
  synthetic primer actin us
cacactgtgcccatctacga

SEQ ID NO: 6 - DNA, 24 bp, Artificial Sequence
  synthetic primer actin ds
tgaggatcttcatgagctagtcag